(19) United States
(12) Patent Application Publication
(10) Pub. No.: US 2023/0170087 A1
(43) Pub. Date: Jun. 1, 2023

(54) SYSTEMS AND METHODS FOR REMOTE PREGNANCY MONITORING AND MANAGEMENT

(71) Applicant: Marani Health, Inc., Oakdale, MN (US)

(72) Inventors: Ann Holder, Stillwater, MN (US); Kathleen Ann Tune, Minneapolis, MN (US); Mark Holm, Rochester, MN (US); Rich Dean Dettinger, Rochester, MN (US); Allison Nicole Schmidt, Las Vegas, NV (US); Masa Vilus, Belgrade (RS); Christian Larson, Minneapolis, MN (US)

(21) Appl. No.: 18/060,761

(22) Filed: Dec. 1, 2022

Related U.S. Application Data
(60) Provisional application No. 63/264,775, filed on Dec. 1, 2021.

(51) Int. Cl. G16H 40/67

(52) U.S. Cl. CPC G16H 40/67

(57) ABSTRACT

In some examples, a system includes a memory; and one or more processors in communication with the memory. The one or more processors are configured to: receive, from a user device, a user input including a request to initiate a physiological data collection procedure; cause the user device to display a first interactive page including one or more instructions for preparing for the physiological data collection procedure; and receive, from the user device, a user input including a request to start the physiological data collection procedure. Additionally, the one or more processors are configured to cause the user device to display a second interactive page including a set of icons, wherein each icon of the set of icons corresponds to a sensor of a set of sensors on a wearable device of the patient.

This application claims the benefit of U.S. Provisional Patent Application No. 63/264,775, filed on Dec. 1, 2021, the entire content of which is incorporated herein by reference.

### TECHNICAL FIELD

The disclosure relates generally to medical device systems and, more particularly, medical device systems configured to monitor patient parameters.

### BACKGROUND

Reliable assessment of fetal well-being is a persistent challenge of current fetal monitoring technologies, including the non-invasive cardiotocography (CTG) technologies and invasive fetal scalp electrodes. The poor specificity and reliability of these techniques have the potential to lead to adverse maternal and fetal outcomes, including unnecessary cesarean sections, related post-surgical complication, inaccurate detection of fetal hypoxia and other fetal complications.

### SUMMARY

In general, the disclosure describes devices, systems, and/or methods for predicting maternal and/or fetal health outcomes based on maternal and/or fetal data. The maternal and/or fetal data (also referred to herein as “patient data”) may include, for example, data regarding sensed biopotential signals such as maternal and/or fetal electrocardiography (ECG) signals, maternal electromyography (EMG) signals, and/or other biopotential signals. The patient data may further include maternal and/or fetal biometric data such as blood pressure, weight, glucose, pH blood levels, blood oxygen level, breathing rate, patient movement, temperature, and other biometric data. In some examples, the patient data may further include data obtained from a mental health assessment, a social determinates of health (SDoH) assessment, socio-economic data for the patient, etc. The patient data may further include any data that may be relevant for the prediction of maternal and/or fetal outcomes, and the disclosure is not limited in this respect.

The techniques may assist clinicians in identification of features or patterns in patient data that could lead to sub-optimal outcomes and support real time decision-making by a clinical team, thus helping to promote timely, appropriate interventions and reducing costs associated with adverse outcomes.

In some examples, according to one or more techniques of the disclosure, a patient computing device executes a patient application in order to display one or more interactive pages related to a patient monitoring session. These pages may include information relating to maternal and/or fetal biopotential data, biometric, or other physiological data. The pages may further include information relating patient health assessment data. Additionally, or alternatively, the one or more interactive pages may include one or more user interaction elements by which a user may interact with patient application. The interactive pages may inform a patient that a monitoring session is due, help the patient proceed through a monitoring session, and prompt a patient to upload data when the monitoring session is complete. The interactive pages may additionally or alternatively allow the patient view physiological data from a recent monitoring session and/or historical physiological data from past tests.

In some examples, according to one or more techniques of the disclosure, a provider device is configured to display one or more interactive pages related to a patient monitoring session. Users of the provider device may include physicians, nurses, technicians, and other clinicians. The interactive pages displayed by the provider device may include information (e.g., maternal and/or fetal biopotential data, biometric data, physiological data, health assessment data, time logs tracking monitoring sessions, etc.) corresponding to a set of patients. The interactive pages may include one or more user interaction elements by which a user may provide user inputs. The interactive pages may allow a clinician to select one or more patients and/or view information relating to one or more patients.

In one example, A system comprises: a memory; and one or more processors in communication with the memory. The one or more processors are configured to: receive, from a user device, a user input including a request to initiate a physiological data collection procedure; cause the user device to display a first interactive page including one or more instructions for preparing for the physiological data collection procedure; receive, from the user device, a user input including a request to start the physiological data collection procedure; and cause the user device to display a second interactive page including a set of icons, wherein each icon of the set of icons corresponds to a sensor of a set of sensors on a wearable device of the patient, and wherein each icon of the set of icons indicates a level of contact between a patient and the respective sensor of the set of sensors.

In another example, A non-transitory computer-readable medium comprising instructions for causing one or more processors to: receive, from a user device, a user input including a request to initiate a physiological data collection procedure; cause the user device to display a first interactive page including one or more instructions for preparing for the physiological data collection procedure; receive, from the user device, a user input including a request to start the physiological data collection procedure; and cause the user device to display a second interactive page including a set of icons, wherein each icon of the set of icons corresponds to a sensor of a set of sensors on a wearable device of the patient, and wherein each icon of the set of icons indicates a level of contact between a patient and the respective sensor of the set of sensors.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a diagram of an example system for the acquisition and communication of patient data and prediction of maternal and/or fetal outcomes using trained machine learning model(s), in accordance with one or more techniques of the disclosure.

FIG. 2 is a more detailed block diagram of the electronic components of an example wearable device and an example patient computing device, in accordance with one or more techniques of the disclosure.

FIG. 3 is a block diagram of an example fetal monitoring system (FMS) in accordance with one or more techniques of the disclosure.

FIG. 4 is a diagram of an example wearable device including a plurality of sensors embedded or affixed to a wearable garment, in accordance with one or more techniques of the disclosure.

FIGS. 5A-5D are graphs illustrating an example mixed maternal-fetal ECG signal (FIG. 5A), a fetal ECG signal extracted from the mixed maternal-fetal ECG signal (FIG. 5B), a graph showing an identification of peaks in the fetal heart rate (FIG. 5C), and a presentation of fetal heart rate as determined based on the fetal ECG signal (FIG. 5D), respectively, in accordance with one or more techniques of the disclosure.

FIGS. 6A-6C are graphs showing an example abdominal (e.g., EMG) signal obtained using the example wearable device 150 as shown in FIG. 4 (FIG. 6A), the envelope of the original uterine activity (UA) and smoothed UA signal (FIG. 6B) and the detected uterine activity signal (FIG. 6C), in accordance with one or more techniques of the disclosure.

FIG. 6D is a combined fetal heart rate and uterine signal graph, in accordance with one or more techniques of the disclosure.

FIG. 7 a conceptual diagram illustrating computing device of FIG. 2, in accordance with one or more techniques of this disclosure.

FIG. 8A is a conceptual diagram illustrating a first test select interactive page, in accordance with one or more techniques of this disclosure.

FIG. 8B is a conceptual diagram illustrating a second test select interactive page 800B, in accordance with one or more techniques of this disclosure.

FIG. 9A is a conceptual diagram illustrating a first health check information interactive page, in accordance with one or more techniques of this disclosure.

FIG. 9B is a conceptual diagram illustrating a second health check information interactive page, in accordance with one or more techniques of this disclosure.

FIG. 9C is a conceptual diagram illustrating a health check initialization interactive page, in accordance with one or more techniques of this disclosure.

FIG. 10A is a conceptual diagram illustrating a first health check sensor contact interactive page, in accordance with one or more techniques of this disclosure.

FIG. 10B is a conceptual diagram illustrating a troubleshooting interactive page that is accessible via the health check sensor contact interactive page of FIG. 10A, in accordance with one or more techniques of this disclosure.

FIG. 10C is a conceptual diagram illustrating an error interactive page, in accordance with one or more techniques of this disclosure

FIG. 10D is a conceptual diagram illustrating a second health check sensor contact interactive page, in accordance with one or more techniques of this disclosure.

FIG. 10E is a conceptual diagram illustrating a third health check sensor contact interactive page, in accordance with one or more techniques of this disclosure.

FIG. 10F is a conceptual diagram illustrating an example of a health check visibility interactive page, in accordance with one or more techniques of this disclosure.

FIG. 10G is a conceptual diagram illustrating a fourth health check sensor contact interactive page, in accordance with one or more techniques of this disclosure.

FIG. 11A is a conceptual diagram illustrating a first health check progress interactive page, in accordance with one or more techniques of this disclosure.

FIG. 11B is a conceptual diagram illustrating a second health check progress interactive page, in accordance with one or more techniques of this disclosure.

FIG. 12A is a conceptual diagram illustrating a first health check upload interactive page, in accordance with one or more techniques of this disclosure.

FIG. 12B is a conceptual diagram illustrating a second health check upload interactive page, in accordance with one or more techniques of this disclosure.

FIG. 12C is a conceptual diagram illustrating a third health check upload interactive page, in accordance with one or more techniques of this disclosure.

FIG. 13A is a conceptual diagram illustrating a first health check results interactive page, in accordance with one or more techniques of this disclosure.

FIG. 13B is a conceptual diagram illustrating a second health check results interactive page, in accordance with one or more techniques of this disclosure.

FIG. 13C is a conceptual diagram illustrating a third health check results interactive page, in accordance with one or more techniques of this disclosure.

FIG. 14 is a conceptual diagram illustrating a nonstress test information interactive page, in accordance with one or more techniques of this disclosure.

FIG. 15A is a conceptual diagram illustrating a first nonstress test initialization interactive page, in accordance with one or more techniques of this disclosure.

FIG. 15B is a conceptual diagram illustrating a second nonstress test initialization interactive page, in accordance with one or more techniques of this disclosure.

FIG. 16A is a conceptual diagram illustrating a first nonstress test sensor contact interactive page, in accordance with one or more techniques of this disclosure.

FIG. 16B is a conceptual diagram illustrating a second nonstress test sensor contact interactive page, in accordance with one or more techniques of this disclosure.

FIG. 16C is a conceptual diagram illustrating a third nonstress test sensor contact interactive page, in accordance with one or more techniques of this disclosure.

FIG. 17 is a conceptual diagram illustrating an example of a nonstress test visibility interactive page, in accordance with one or more techniques of this disclosure.

FIG. 18A is a conceptual diagram illustrating a first nonstress test progress interactive page, in accordance with one or more techniques of this disclosure.

FIG. 18B is a conceptual diagram illustrating a nonstress test progress interactive page, in accordance with one or more techniques of this disclosure.

FIG. 19 is a conceptual diagram illustrating a nonstress test upload interactive page, in accordance with one or more techniques of this disclosure.

FIG. 20 is a conceptual diagram illustrating a nonstress test complete interactive page, in accordance with one or more techniques of this disclosure.

FIG. 21A is a conceptual diagram illustrating a maternal heart rate patient data history interactive page, in accordance with one or more techniques of this disclosure.

FIG. 21B is a conceptual diagram illustrating a nonstress test history interactive page, in accordance with one or more techniques of this disclosure.

FIG. 22 is a conceptual diagram illustrating an interactive page including a list of patients for display by provider portal, in accordance with one or more techniques of this disclosure.

FIG. 23 is a conceptual diagram illustrating an interactive page including data corresponding to a patient monitoring session, in accordance with one or more techniques of this disclosure.

FIG. 24 is a conceptual diagram illustrating an interactive page including data corresponding to a set of nonstress tests, in accordance with one or more techniques of this disclosure.

FIG. 25 is a conceptual diagram illustrating an interactive page including data corresponding to a set of patient glucose level tests, in accordance with one or more techniques of this disclosure.

FIG. 26 is a conceptual diagram illustrating an interactive page including data corresponding to a set of patient blood pressure tests, in accordance with one or more techniques of this disclosure.

FIG. 27 is a conceptual diagram illustrating an interactive page including data corresponding to a patient's weight, in accordance with one or more techniques of this disclosure.

FIGS. 28-30 are conceptual diagrams illustrating interactive pages for display by patient computing device, in accordance with one or more techniques of this disclosure.

FIG. 31 is a flow diagram illustrating an example operation for using an application to ensure a good connection between sensors of a medical device and a patient, in accordance with one or more techniques of this disclosure.

### DETAILED DESCRIPTION

In general, the disclosure describes devices, systems, and/or methods for predicting maternal and/or fetal health outcomes based on maternal and/or fetal data. The maternal and/or fetal data (also referred to herein as “patient data”) may include, for example, data regarding sensed biopotential signals such as maternal and/or fetal electrocardiography (ECG) signals, maternal electromyography (EMG) signals, and/or other biopotential signals. The patient data may further include maternal and/or fetal biometric data such as blood pressure, weight, glucose, pH blood levels, blood oxygen level, breathing rate, patient movement, temperature, and other biometric data. In some examples, the patient data may further include data obtained from a mental health assessment, a social determinates of health (SDoH) assessment, socio-economic data, etc. The patient data may further include any data that may be relevant for the prediction of maternal and/or fetal outcomes, and the disclosure is not limited in this respect.

The techniques may assist clinicians in identification of features or patterns in patient data that could lead to sub-optimal outcomes, support real time decision-making by the clinical team, thus helping to promote timely, appropriate interventions, and decrease overall costs associated with adverse maternal and fetal outcomes. The techniques may aid clinicians and healthcare providers to improve prenatal care and to better manage risk pregnancy patients while at home, allowing for continued monitoring and alert triggering. In addition, healthcare costs associated with pregnancy may be reduced by eliminating unnecessary travels and clinic visits, saving time and stress to future mothers. In addition, collection of relevant patient data may provide a framework for clinical and scientific research in the field of prenatal care and support continuous updates and refinements to the predictive models and the resulting predicted maternal and/or fetal outcomes.

In some examples, according to one or more techniques of the disclosure, a training data set including patient data and associated outcomes obtained for each of a plurality of patients (e.g., pregnant human mothers and their fetuses) is used to train a machine learning model for maternal and/or fetal outcome prediction. The machine learning model is indicative of features of the patient data are predictive of one or more maternal or fetal outcomes (either adverse or non-adverse).

In some examples, according to one or more techniques of the disclosure, a cloud-based pregnancy monitoring system receives patient data associated with a pregnant mother, applies the patient data to the trained machine learning model, and predicts one or more fetal and/or maternal outcomes associated with the pregnant mother based on the patient data.

Although specific examples using maternal and/or fetal ECG or heart rate data to predict one or more outcomes are described herein, it shall be understood that the disclosure also applies to prediction of outcomes using any other type of patient data, including other sensed biopotential signals, biometric data, socio-economic data, mental health data or any other data relevant to prediction of maternal and/or fetal outcomes, and that the disclosure is not limited in this respect.

The techniques of the disclosure may predict and output one or more maternal and/or fetal outcomes. Predicted fetal outcomes may include, but are not limited to, Apgar scores (e.g., 1, 5 and 10 minutes after birth), cord blood gas pH level, neonatal destination immediately after birth, admission to Neonatal Intensive Care Unit (NICU) within 48 hours of birth, NICU length of stay, resuscitation intervention, other neonatal complications, neonatal death up to 28 days after birth, etc. Predicted maternal outcomes may include, but are not limited to, mode of delivery (e.g., vaginal or C-section), reason for C-section, grade of C-section (If performed—Grades 1, 2, 3 or 4), length of stay, destination immediately after birth, admission to a higher level of care, complications (type and severity), hour of day of delivery, day of week of delivery, etc.

In some examples, one or more techniques of the disclosure combine patient data such as maternal and/or fetal ECG or heart rate data with additional patient data including biometric data such as uterine contraction data, blood pressure, weight, glucose, pH blood levels, blood oxygen level, breathing rate, patient movement, temperature; patient health assessment data such as results of a mental health assessments, a social determinates of health (SDoH) assessment, data regarding preexisting conditions, patient usage patterns (for example, the timing or update patterns when answering questions on a psychological survey), time of day, frequency or time between measurements, and/or any other patient data relevant to prediction of maternal and/or fetal outcomes for use as training data and/or input data for a current monitoring session for which one or more outcomes are predicted.

The training data may be used to generate one or more ML models for the identification of high-risk pregnancies (e.g., prediction of one or more adverse outcomes described herein). The techniques of the disclosure may help identify false predictions of fetal distress that may lead to unnecessary Cesarean sections, so that unnecessary C-Sections and the associated increase in health care costs and maternal recovery time may be minimized. At the same time, accuracy regarding the prediction of actual fetal distress may be maximized, allowing for timely interventions when needed. The techniques of the disclosure thus provide a comprehensive and accurate monitoring system that takes many types, attributes, features, and/or patterns of fetal and/or maternal data into account when predicting one or more maternal and/or fetal outcomes.

In some examples, the techniques of the disclosure include a wearable device for acquiring maternal and/or fetal biopotential (such as ECG and/or EMG) or heart rate data that a pregnant mother can use at home or other non-clinical environment, which in combination with a cloud-based remote monitoring system (e.g., telehealth and/or telemedicine system), may improve the mother's comfort and peace of mind during pregnancy. The techniques may be used to monitor the health of prenatal and postpartum patients in a remote monitoring setting. The techniques of the disclosure may also be used during labor and delivery in addition to or instead of a traditional cardiotocography (CTG) monitoring device in clinical/hospital environment.

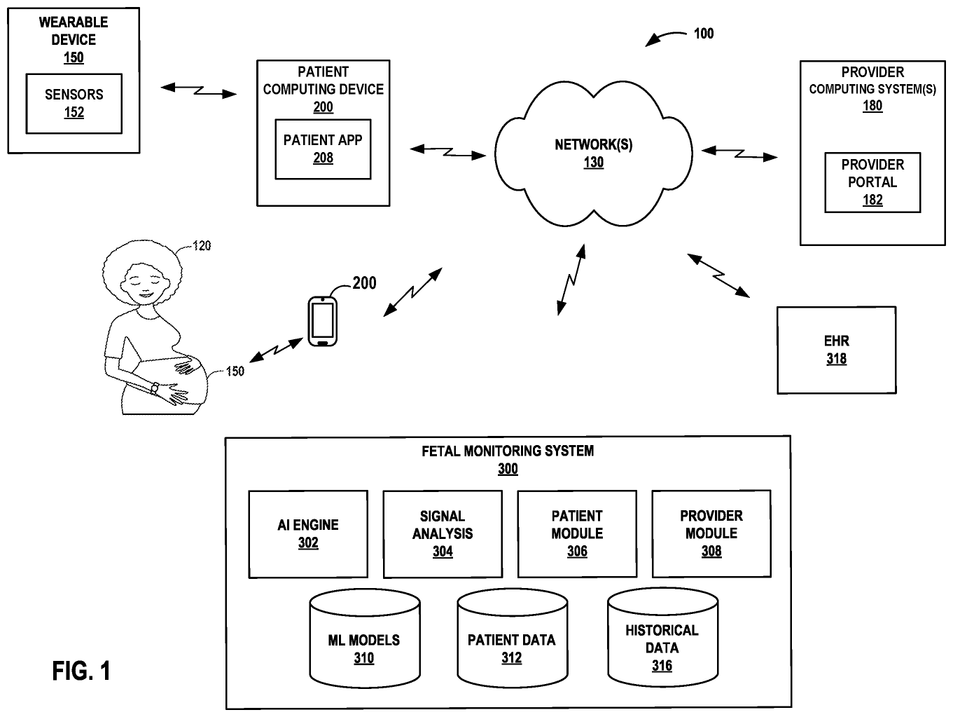

FIG. 1 is a diagram of an example system 100 for the acquisition and communication of patient data and/or prediction of maternal and/or fetal outcomes using trained machine learning model(s) in accordance with one or more techniques of the disclosure. In this example, system 100 includes a wearable device 150 including a plurality of sensors 152 (e.g., electrodes) configured to sense physiological signals of a patient 120 (a pregnant human mother and/or her fetus). The physiological signals may include, for example, maternal and/or fetal biopotential signals, such as ECG signals or other signals indicative of maternal and/or fetal cardiac activity, EMG signals indicative of uterine activity or contractions, or any other sensed physiological data. System 100 further includes at least one patient computing device 200, provider computing system 180, and a cloud-based fetal monitoring system (FMS) 300.

In some examples, wearable device 150 includes a wearable (e.g., a garment or a band) configured to be worn about the torso of a pregnant patient 120, one or more sensors 152 affixed or embedded in the wearable, a communications interface, and a controller. The one or more sensors 152 are configured to sense physiological signals, such as one or more biopotential signals of the mother and/or the fetus, such as ECG and/or EMG signals. In some examples, the sensed physiological data includes maternal and/or fetal ECG or heart rate data; however, the disclosure is not limited in this respect. Wearable device 150 is configured to wirelessly communicate sensor data representative of the sensed physiological signals for receipt by at least one computing device, such as patient computing device 200. The wearable device controller is configured to control signal acquisition from the one or more sensors and to control wireless communication of the sensor data.

Patient computing device 200 is configured for wireless communication with wearable device 150. For example, patient computing device 200 wirelessly receives the sensor data transmitted by wearable device 150. In some examples, patient computing device 200 may include one or more personal computing devices of the patient 120. For example, patient computing device 200 may include a mobile computing device (e.g., smartphone, tablet, or laptop computer), a desktop computer, a smartwatch, etc. Computing device 200 and wearable device 150 may communicate using, for example, the Bluetooth® or Bluetooth® Low Energy (BLE) protocols, near field communication (NFC), Wi-Fi, or any other form of wireless and/or wired communication.

In some examples, patient computing device 200 includes a patient application 208 stored in a memory or other data storage device of patient computing device 200 as a computer-readable medium comprising instructions that, when executed by patient computing device 200, generates one or more interactive pages for display on a user interface of patient computing device 200. The one or more interactive pages guide the patient through a monitoring session during which physiological signals are acquired by wearable device 150 and corresponding sensed patient data is communicated from wearable device 150 to patient computing device 200. Example systems and methods for remote pregnancy monitoring and management are shown and described in U.S. Patent Application No. 63/264,775, filed Dec. 1, 2022, which is incorporated by reference herein in its entirety.

Patient computing device 200 is further configured to communicate with a variety of other devices or systems via network(s) 130. For example, computing device 200 may be configured to communicate with one or more computing systems, e.g., provider computing system 180 and/or Fetal Monitoring System (FMS) 300.

FMS 300 includes an AI engine 302, a signal analysis module 304, a patient module 306, and a provider module 308. FMS 300 further includes or is associated with one or more databases or other storage device(s) that store one or more stored machine learning (ML) model(s) 310, patient data 312, sensor data 314, and historical data 316. Sensor data 314 includes the raw data representative of the biopotential signals detected by wearable device 150 during one or more patient monitoring sessions. Patient data 312 includes, for each of a plurality of patients, identification information corresponding to the patient, processed sensor data analyzed or generated by FMS 300 corresponding to one or more patient monitoring sessions, and/or one or more predicted outcomes corresponding to the one or more patient monitoring sessions. Historical data 316 includes historical maternal and/or fetal patient data associated with a plurality of patients. FMS 300 executes provider module 308 to provide remote provider-facing fetal monitoring services that support healthcare provider interaction with FMS 300 via provider portal 182 of provider computing system(s) 180. Similarly, FMS 300 executes patient module 306 to provide remote patient-facing fetal monitoring services that support patient interaction with FMS 300 via patient application 208 of patient computing device 200.

In accordance with one or more techniques of the disclosure, AI engine 302 of FMS 300 is configured to train one or more machine learning (ML) model(s) 310 based on historical data 316 associated with a plurality of patients to generate one or more maternal and/or fetal outcome predictions. AI engine 302 is further configured to determine, based on processing patient data for a pregnant patient using one or more ML models 310 trained with the historical data 316, one or more maternal and/or fetal outcome predictions for the pregnant patient. Example systems and methods of training of the one or more machine learning models and or prediction of one or more maternal and/or fetal outcomes are described in U.S. patent application Ser. No. 17/457,206, filed Dec. 1, 2021, U.S. Provisional Patent Application 63/265,952, filed Dec. 23, 2021, and U.S. Provisional Patent Application 63/268,244, filed Feb. 18, 2022, each of which is incorporated by reference herein in its entirety.

Patient computing device(s) 200 may transmit data, including patient data received from wearable device 150, to computing system(s) 180 and/or FMS 300 via network(s) 130. The data may include sensed patient data, e.g., values of one or more biopotential signals, such as ECG and/or EMG signals, sensed by wearable device 150 and other physiological signals or data sensed or otherwise determined by wearable device 150 and/or patient computing device(s) 200. FMS 300 may retrieve data regarding patient(s) from one or more sources of electronic health records (EHR) 318 (which may also be referred to as electronic medical records, EMR) via network 130. EHR 318 may include data regarding historical (e.g., baseline) patient data, previous health events and treatments, preexisting conditions, disease states, comorbidities, demographics, height, weight, and body mass index (BMI), as examples, of patients. FMS 300 may use data from EHR 318 to configure algorithms implemented by wearable device 150, patient computing device 200 and/or FMS 300 to control acquisition of the sensed biopotential signals from wearable device 150 during a monitoring session and/or to predict maternal and/or fetal outcomes based on patient data acquired during a monitoring session for a patient.

Network(s) 130 may include, for example one or more local area networks (LANs), wireless local area networks (WLANs), virtual private networks (VPNs), wide area networks (WANs), the Internet, etc. Network(s) 130 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network(s) 130 may include one or more networks administered by service providers and may thus form part of a large-scale public network infrastructure, e.g., the Internet. Network(s) 130 may provide computing devices and systems, such as those illustrated in FIG. 1, access to the Internet, and may provide a communication framework that allows the computing devices and systems to communicate with one another. In some examples, network(s) 130 may include a private network that provides a communication framework that allows the computing devices and systems illustrated in FIG. 1 to communicate with each other but isolates some of the data flows from devices external to the private network for security purposes. In some examples, the communications between the computing devices and systems illustrated in FIG. 1 are encrypted.

Provider computing system 180 includes one or more computing devices used by providers (e.g., physicians, physician assistants, nurses, nurse midwives, pharmacists, therapists, clinical support staff, etc.) to view patient data gathered or generated during one or more patient monitoring sessions, including one or more maternal and/or fetal outcome predictions associated with the patient monitoring sessions, for one or more patients. For example, provider computing system 180 may include a provider portal 182 stored in a memory or other data storage device of provider computing system 180 as a computer-readable medium comprising instructions that when executed by provider computing system 180 generates one or more interactive pages for display on a user interface of provider computing system 180 that allow health care providers to view raw and/or processed patient data or other data generated by analysis of the patient data, including one or more predicted maternal and/or fetal outcomes, for one or more patients.

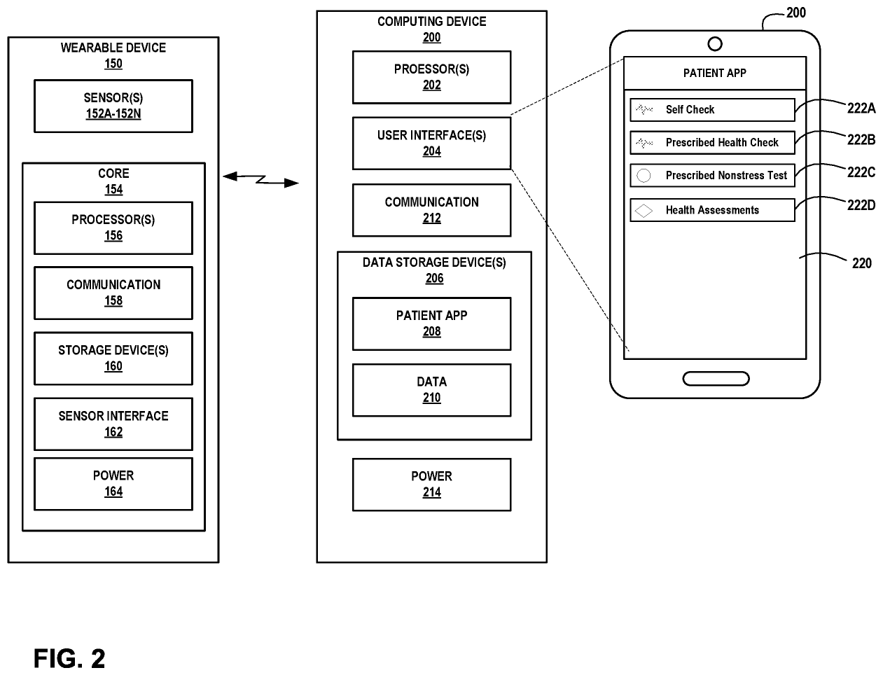

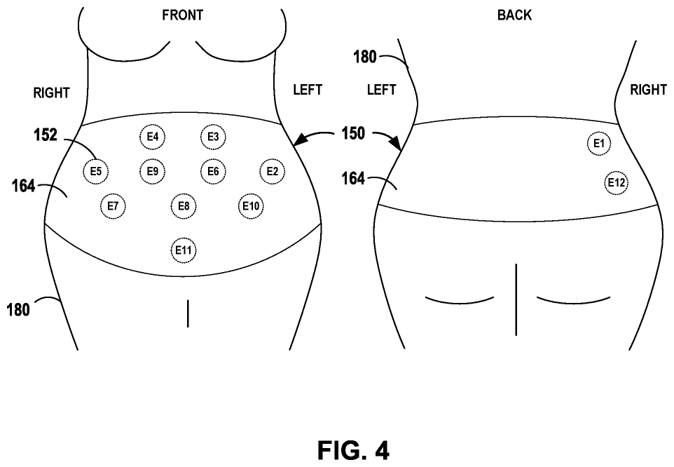

FIG. 2 is a more detailed block diagram of the electronic components of an example wearable device 150 and an example patient computing device 200 in accordance with one or more techniques of the disclosure. Wearable device 150 includes one or more sensors 152 (e.g., electrodes) configured to sense physiological signals of a patient, such as maternal and/or fetal biopotential signals, such as ECG and/or EMG signals. In some examples, wearable device 150 includes a wearable (e.g., a garment or band 164 such as shown in FIG. 4) including a plurality of electrodes or other sensing devices 152A-152N affixed to or embedded therein.

In some examples, the sensors are configured to sense at least one of maternal or fetal biopotential signals, such as at least one of maternal or fetal ECG signals. In other examples, one or more of the sensors may be configured to sense any one or more of cardiotocography (CTG) signals, electromyography (EMG) signals, EMG myometrium signals, pulse oximeter signals, respiratory inductance plethysmography (RIP) (thoracic and abdominal) signals, acoustic signals, actigraphy signals, temperature information (temperature sensor(s)), accelerometer or movement information, photoplethysmography (PPG) (e.g., optical measurement for pulse rate and SpO2), and/or any other biopotential or physiological signal or parameter of the patient. The sensors 152 may thus further include any appropriate sensor(s) configured to detect or sense any of the listed signals or physiological parameter associated with the patient.

Wearable device further includes control electronics that process the sensed physiological signals of the patient acquired by sensors 152 and communicate the sensed patient data for receipt by patient computing device 200. In some examples, the control electronics are packaged in a core 154 configured to be removably connected to the wearable garment or band. To that end, core 154 includes one or more processors 156, a communication interface 158, storage devices 160, a sensor interface 162, and a power source 164 (e.g., one or more batteries). Sensor interface 162 includes circuitry configured to receive sensor data corresponding to the physiological signals detected by the one or more sensors 152. Communication interface 158 is configured to support wireless communication between wearable device 150 and one or more computing devices, such as patient computing device 200. Storage devices 160 include one or more hardware memories or other data storage devices configured to store executable control instruction and/or raw sensor data associated with one or more monitoring sessions. Wearable device 150 may store sensor data temporarily during each monitoring session for wireless transmission to a computing device, or wearable device may store sensor data associated with multiple monitoring sessions for later transmission to a computing device.

Patient computing device 200 includes one or more processor(s) 202, a user interface 204, communication interface 212, data storage devices 206, and a power source 214 (e.g., one or more batteries). In some examples, patient computing device 200 may include one or more personal computing devices of the patient. For example, patient computing device 200 may include a mobile computing device (e.g., smartphone, tablet, or laptop computer), a desktop computer, a smartwatch, etc. Communication interface 212 of patient computing device 200 is configured for wireless communication with wearable device 150. For example, communication interface 212 and communication interface 158 of wearable device 150 may be configured to communicate using, for example, the Bluetooth® or Bluetooth® Low Energy (BLE) protocols, near field communication (NFC), or any other form of wireless communication.

Patient computing device 200 includes a patient application 208 stored in data storage device(s) 206. For example, patient application 208 may include a computer-readable medium comprising instructions that, when executed by one or more processor(s) 202 of patient computing device 200, generates one or more interactive pages for display on a user interface 204 of patient computing device 200 that guide the patient through a monitoring session during which physiological signals are acquired by wearable device 150 and corresponding sensor data is communicated from wearable device 150 to patient computing device 200. As shown in the example of FIG. 2, example patient computing device 200 includes a touch screen display 220 on which one or more interactive pages of a guided patient monitoring session are displayed. Each interactive page may include one or more user interface elements, such as user interface elements 222A-222D, by which a user may interact with patient application 208 (and thus with wearable device 150 and/or FMS 300) to conduct one or more monitoring sessions including a self-check, a prescribed health check, a prescribed nonstress test, and/or one or more health assessments, such as one or more mental health assessments, social determinants of health assessments, socio-economic assessments, etc. Example systems and methods for remote pregnancy monitoring and management are shown and described in U.S. Patent Application No. 63/264,775, filed Dec. 1, 2022, which is incorporated by reference herein in its entirety.

Communication interface 204 of patient computing device 200 is further configured to communicate with a variety of other devices or systems via network(s) 130 (see FIG. 1). For example, computing device 200 may be configured to communicate with one or more computing systems, e.g., one or more of provider computing system180 and/or FMS 300.

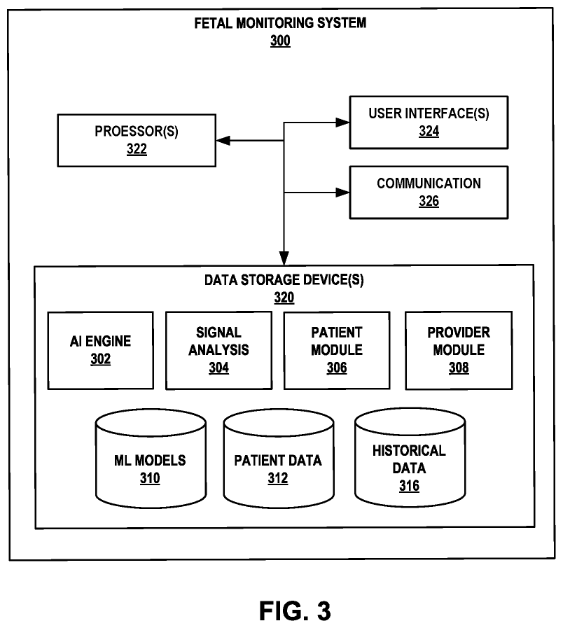

FIG. 3 is a block diagram of an example fetal monitoring system (FMS) 300 in accordance with one or more techniques of the disclosure. FMS 300 includes one or more processors 322, user interfaces 324 by which one or more users may interact with FMS 300, communication interfaces 326 which provide for communication with one or more computing devices such as patient computing device 200 and/or provider computing systems 180, and one or more data storage devices 320. Data storage devices 320 include storage for one or more computing modules including AI engine 302, signal analysis module 304, patient module 306, and provider module 308. FMS 300 further includes or is associated with one or more databases or other storage device(s) that store one or more stored machine learning (ML) model(s) 310, patient data 312, and historical data 316. Patient data 312 includes, for each of a plurality of patients, biopotential or other physiological patient data sensed by wearable device 150 during one or more patient monitoring sessions, biometric data associated with the patient, and/or patient data obtained during one or more health assessment sessions. Patient data 312 also includes, for each of a plurality of patients, identification information corresponding to the patient, processed sensor data analyzed or generated by FMS 300 corresponding to one or more patient monitoring sessions, and/or one or more predicted outcomes corresponding to the one or more patient monitoring sessions. Historical data 316 includes historical patient data associated with a plurality of patients. Processor(s) 322 of FMS 300 execute provider module 308 to provide remote provider-facing fetal monitoring services that support healthcare provider interaction with FMS 300 via provider portal 182 of provider computing system(s) 180. Similarly, processors 322 of FMS 300 execute patient module 306 to provide remote patient-facing fetal monitoring services that support patient interaction with FMS 300 via patient application 208 of patient computing device 200.

Signal analysis module 304 may apply one or more signal processing or preprocessing techniques to the raw sensor data representative of the maternal and/or fetal biopotential signals acquired by the one or more sensors. For example, signal analysis module 304 may apply normalization, denoising, filtering, artifact detection and/or artifact correction to any one or more of the sensed signal data received from the wearable device 150. Signal analysis modules may also perform feature extraction for the sensed biopotential signals including for example, extraction of a fetal ECG signal from a mixed maternal-fetal ECG signal, identification of one or more features of the maternal and/or fetal ECG signals including, for example, one or more features of the P wave, QRS complex, T wave, PQ interval, QRS duration, QT interval, RR interval, or other feature indicative of the electrical activity of the heart (e.g., start, end, duration, amplitude, peak-to-peak information, morphology, etc.). Signal analysis module 304 may further extract one or more features of the maternal and/or fetal heart rate signals including but not limited to, for example, baseline heart rate, baseline variability, fetal heart rate variability, number of accelerations per second, number of early, late, and variable decelerations per second, number of prolonged decelerations per second, sinusoidal pattern, etc.

FMS 300 executes provider module 308 to provide remote provider-facing fetal monitoring services that support healthcare provider interaction with FMS 300 via provider portal 182 of provider computing system(s) 180. Similarly, FMS 300 executes patient module 306 to provide remote patient-facing fetal monitoring services that support patient interaction with FMS 300 via patient application 208 of patient computing device 200.

In accordance with one or more techniques of the disclosure, AI engine 302, when executed by processors 322 of FMS 300, is configured to train one or more machine learning (ML) model(s) 310 based on historical data 316 associated with a plurality of patients to generate one or more maternal and/or fetal outcome predictions. AI engine 302, when executed by processors 322, is further configured to determine, based on processing patient data for a pregnant patient using one or more ML models 310 trained with the historical data 316 corresponding to a plurality of patients, one or more maternal and/or fetal outcome predictions for the pregnant patient.

Although in the examples described herein FMS 300 is described as performing the training of the ML models 310 and/or application of the models 310 to predict one or more maternal or fetal outcomes, some or all of the functions described herein as being performed by FMS 300 may be performed by any one or more of wearable device 150, patient computing device 200, provider computing system 180, or any other remote, local or distributed computing device or system, and that the disclosure is not limited in this respect. In addition, the various functions performed by FMS 300 may be implemented using a single computing device or system or they may be distributed across multiple computing devices or systems.

FIG. 4 is a diagram of an example wearable device 150 including a plurality of sensors 152A-152N (labeled E1-E12 and referred to generally as sensors 152) embedded or affixed to a wearable garment 164 in accordance with one or more techniques of the disclosure. In this example, the sensor configuration includes a total of twelve electrodes E1-E12 affixed to or embedded within garment 164 such that, when properly worn about the torso of a patient 120, the electrodes 152 are positioned about the torso of patient 120. One or more of the electrodes 152 may be positioned on the front, left side, right side and/or back of the patient 120. As shown in FIG. 4, in some examples, the sensor configuration on garment 164 is such that two electrodes are positioned on the back of patient 120 (E1 and E12 in this example). The number and configuration of sensors 152 on the wearable garment 164 may vary from that shown in FIG. 4, and the disclosure is not limited in this respect.

In order to capture maternal and fetal biopotential signals of sufficient quality, sensors 152 should provide good contact with the patient's skin, minimize sensor movement relative to the skin, and reduce signal noise from light movements of the patient. In some examples, one or more of sensors 152 include SilverBumps® dry electrodes available from Orbital Research, Inc. Example wearable garments that may be used to implement wearable device 150 are described in U.S. Pat. 9,579,055, issued Feb. 28, 2017, which is incorporated by reference herein in its entirety.

In other examples, instead of or in addition to dry electrodes, wearable device 150 may include any other type of sensing material or device to acquire the biopotential signals data, such as one or more of nanotechnology sensing devices, textile or silicon-based dry electrodes, nanotube sensors, cardiotocography (CTG) doppler transducers for acquiring signals associated with uterine contractions, and/or any other sensor that may be used to capture maternal and/or fetal biopotential signals.

In accordance with one or more techniques of the disclosure, the physiological (e.g., biopotential) signals sensed by wearable device 150 and analyzed to determine the status of the fetus and/or predict one or more maternal and/or fetal outcomes may include, but are not limited to, fetal heart rate (fHR), maternal heart rate (mHR), fetal ECG, maternal ECG, and maternal EMG signals.

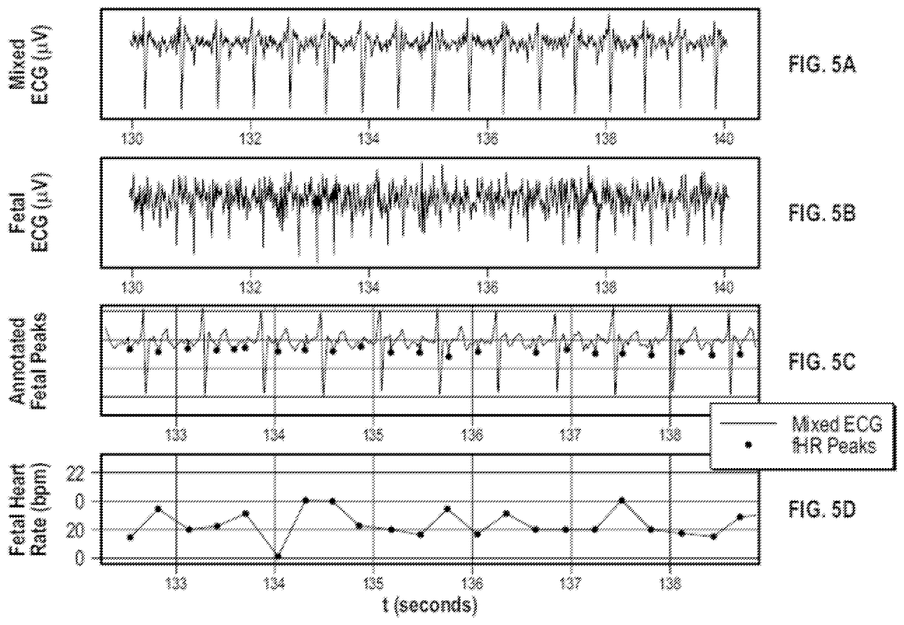

FIGS. 5A-5D are graphs illustrating an example mixed maternal-fetal ECG signal (FIG. 5A), a fetal ECG signal extracted from the mixed maternal-fetal ECG signal (FIG. 5B), a graph showing an identification of peaks in the fetal heart rate (FIG. 5C), and a presentation of fetal heart rate as determined based on the fetal ECG signal (FIG. 5D), respectively, in accordance with one or more techniques of the disclosure.

To obtain the fetal ECG (FIG. 5B) from the mixed (maternal and fetal) ECG signal (FIG. 5A) that is captured by the wearable device, an extraction algorithm may be employed. Example techniques for extracting a fetal ECG signal (FIG. 5B) from a mixed maternal-fetal ECG signal are described in U.S. Pat. No. 11,224,375, issued Jan. 18, 2022, which is incorporated by reference herein in its entirety.

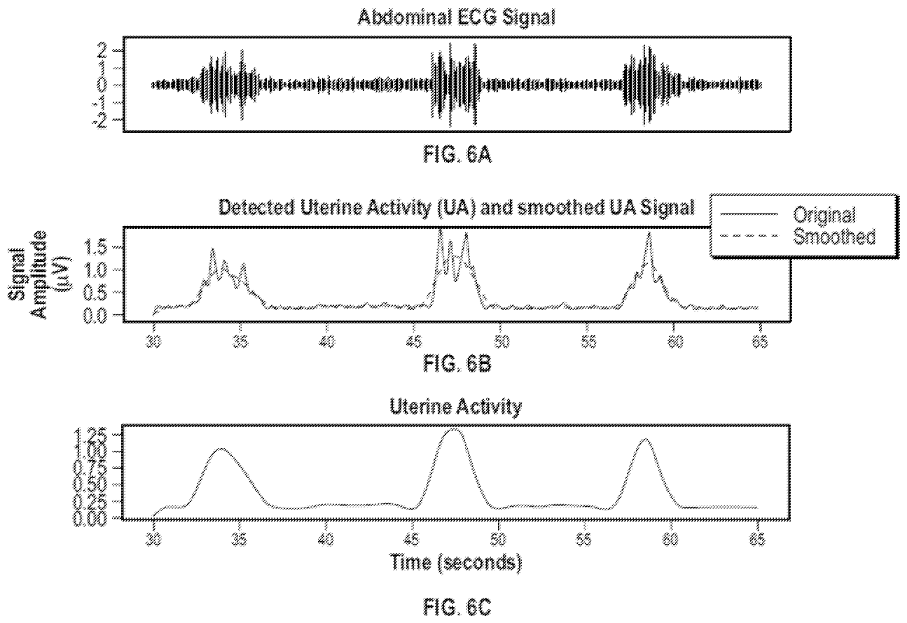

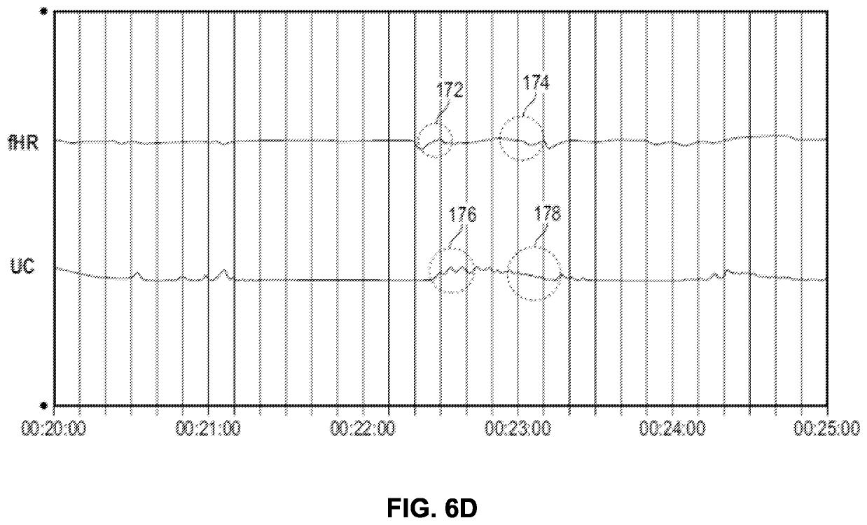

FIGS. 6A-6C are graphs showing an example abdominal (e.g., EMG) signal obtained using the example wearable device 150 as shown in FIG. 4 (FIG. 6A), the envelope of the original uterine activity (UA) and smoothed UA signal (FIG. 6B) and the detected uterine activity signal (FIG. 6C), in accordance with one or more techniques of the disclosure.

FIG. 6D is a combined fetal heart rate and uterine signal graph in accordance with one or more techniques of the disclosure. Example features of the fetal heart rate signal (upper portion of the graph) are indicated by reference numerals 172 and 174 and example features of the uterine contraction signal (lower portion of the graph) are identified by reference numerals 176 and 178. Reference numeral 172 indicates an acceleration of the fetal heart rate signal that occurred during a first period of time. Reference numeral 174 indicates a deceleration of the fetal heart rate signal that occurred during a second period of time. The detected accelerations/decelerations of the fetal heart rate signal (or the average, mean or other statistical characterization of the detected accelerations/decelerations) may be extracted as a feature that is input to the ML models for the prediction of maternal and/or fetal outcomes. Reference numeral 176 indicates the start of a uterine contraction substantially corresponding to the first period of time during which the fetal heart acceleration indicated by reference numeral 172 occurred. Reference numeral 178 indicates the end of the uterine contraction substantially corresponding to the second period of time during which the fetal heart rate deceleration 174 occurred.

The system may extract one or more features of the sensed biopotential signals and use the extracted features as inputs to a machine learning model (such as ML model(s) 310) to predict one or more maternal and/or fetal outcomes. For example, features of the fetal heart rate may include, but are not limited to, baseline heart rate, baseline variability, number of accelerations per second, number of early, late, and variable decelerations per second, number of prolonged decelerations per second, sinusoidal pattern, etc. Features of the fetal ECG may include, for example, one or more features of the P wave, QRS complex, T wave, PQ interval, QRS duration, QT interval, RR interval, or other feature indicative of the electrical activity of the heart (e.g., start, end, duration, amplitude, peak-to-peak information, morphology, etc.). In another example, analysis of the raw fetal ECG signal may be considered as well to avoid the information loss associated with such feature extraction procedures.

Similar features may also be identified for the maternal heart rate. Uterine contraction (UC) features may include baseline uterine tone, contraction frequency, start/end time of uterine contractions, amplitude of uterine contractions, duration of uterine contractions, and strength (intensity) of uterine contractions.

Example features of the fetal heart rate may include, but are not limited to, the features shown in Table 1. Similar features may also be identified with respect to the maternal heart rate.

TABLE 1
Variable Description (fHR)

Fetal heart rate baseline (beats per minute)
Number of accelerations
Number of fetal movements
Number of uterine contractions
Number of moderate decelerations
Number of severe decelerations
Number of prolonged decelerations
Percentage of time with abnormal short-term variability
Mean duration of short-term variability
Mean duration of long-term variability
Percentage of time with abnormal long-term variability
Histogram tendency
Fetal state class code (N = Normal, S = Suspected, P = Pathological)
Width of FHR histogram
Minimum of FHR histogram
Maximum of FHR histogram
Number of histogram peaks
Number of histogram zeroes
Histogram mode
Histogram median
Histogram variance
Amplitude of FHR

The patient data for a particular patient may include patient data obtained during one or more previous monitoring sessions for the patient. The patient data associated with the previous monitoring sessions may thus be used to establish one or more baselines for the patient. For example, baselines with respect to maternal ECG and/or heart rate, fetal ECG and/or heart rate, etc., may be established and used as feature inputs to one or more ML models for prediction of maternal and/or fetal outcomes for the patient. In this way, longitudinal information for the patient over time may be taken into account when determining the one or more maternal and/or fetal outcome predictions for the patient.

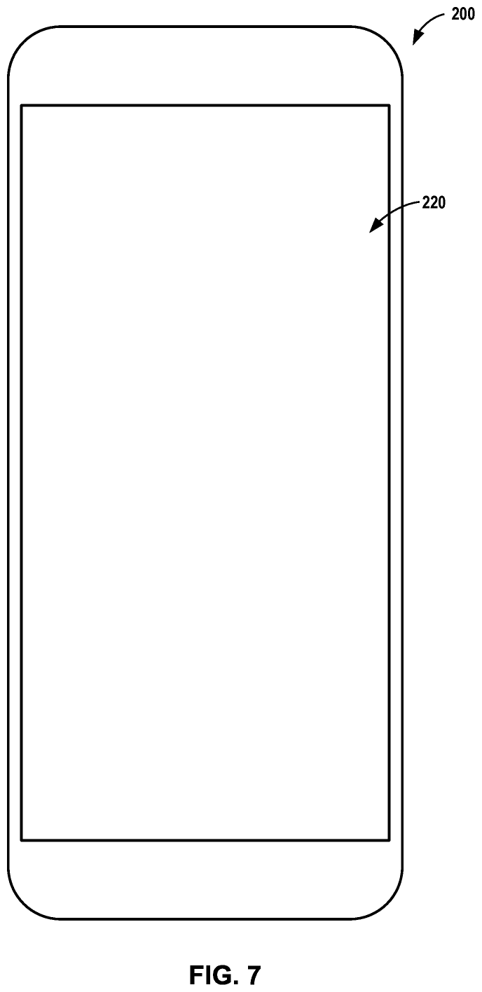

FIG. 7 is a conceptual diagram illustrating a computing device 200, in accordance with one or more techniques of this disclosure. As seen in FIG. 7, computing device 200 includes a screen 220. In some examples, screen 220 represents a touch screen configured to receive one or more user touch inputs. In some examples, computing device 200 may represent a smart phone, but this is not required. Computing device 200 may represent any kind of device that includes a screen for displaying information. Computing device 200 may store a patient application (e.g., patient application 208 of FIG. 2) in a memory. When computing device 200 may executes patient application 208, computing device 200 may display one or more interactive pages on screen 220. In some examples, the term “interactive page” may refer to any page displayed by computing device 200 in the course of executing patient application 208. An interactive page may include one or more interactive elements and/or one or more visual elements. Computing device 200 may perform one or more actions in response to a user input to an interactive element, whereas visual elements may convey information on screen 220 without providing the option for computing device 200 to perform actions based on user input.

In some examples, patient application 208 of FIG. 2 may include one or more series of interactive pages for controlling a wearable device (e.g., wearable device 150 of FIGS. 1-2). For example, wearable device 150 may be configured to collect data for performing one or more tests, such as a health check and a nonstress tests. Wearable device 150 may collect physiological data corresponding to a mother and/or a fetus. By analyzing the physiological data collected by wearable device 150, computing device 200 and/or FMS 300 of FIG. 3 may perform the one or more tests. To perform the one or more tests, it may be beneficial for the patient to follow specific steps or procedures. Computing device 200 may execute patient application 208 in order to display one or more interactive pages on screen 220. These interactive pages may assist the patient in performing the steps necessary to complete the one or more tests.

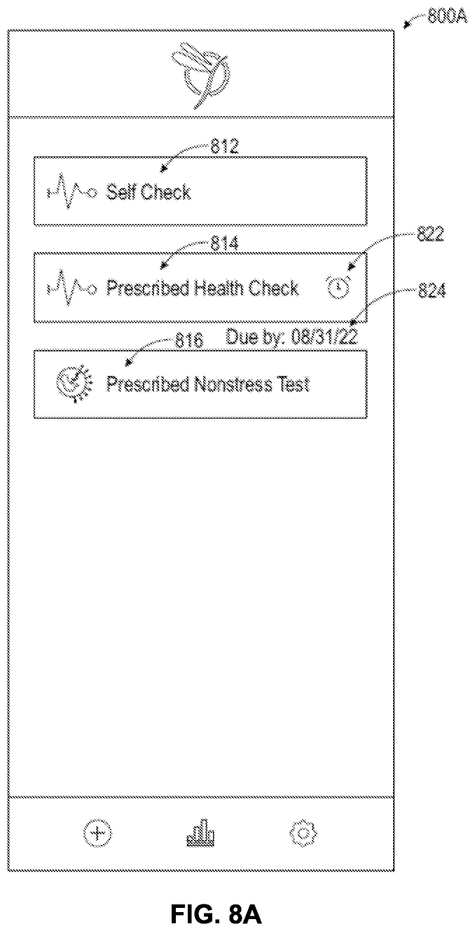

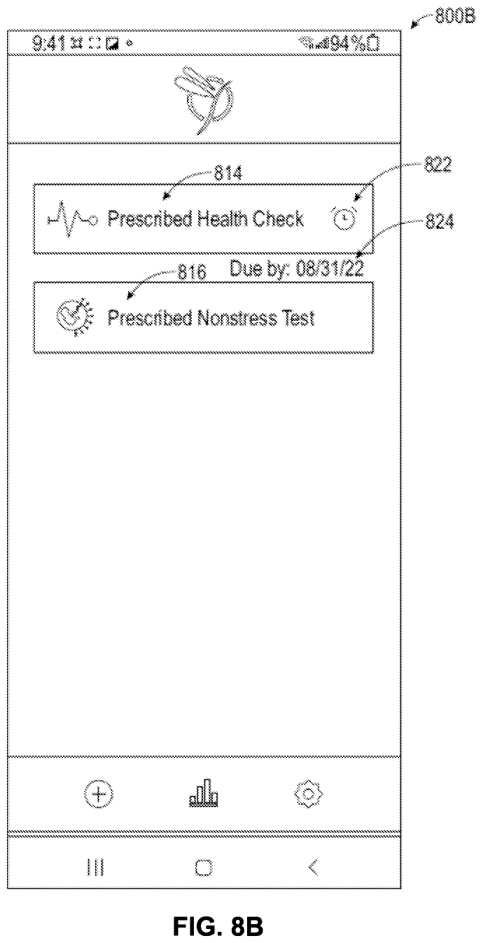

FIG. 8A is a conceptual diagram illustrating a first test select interactive page 800A, in accordance with one or more techniques of this disclosure. As seen in FIG. 8A, test select interactive page 800A includes a first user interaction element 812, a second user interaction element 814, and a third user interaction element 816. Test select interactive page 800A further includes a clock visual element 822 and a deadline visual element 824.

In some examples, first test select interactive page 800A may represent an interactive page that prompts a patient to select either a “self check” or a “prescribed health check.” Self checks and prescribed health checks may be examples of “patient monitoring sessions” described herein. In some examples, a prescribed health check may represent a health check that is prescribed to the patient by a clinician according to a set schedule. In some examples, a clinician may prescribe a set of health checks to the patient, where each health check of the set of health checks is prescribed for a particular time of day and/or a particular day. For example, the clinician may instruct the patient to perform one prescribed health check per day. The clinician may, in some examples, instruct the patient to perform health checks at a particular time of day, but this is not required. First test select interactive page 800A may indicate a day and/or a time that the next prescribed health check is due, display a message if the patient misses a prescribed health check. In some examples, a self-check may represent a health check that the patient decides to conduct, even though the health check was not prescribed or otherwise instructed by a clinician. In some examples, first test select interactive page 800A may display other information relating to self checks and/or prescribed health checks.

First user interaction element 812 may include a selectable “Self Check” option, and second user interaction element 814 may include a selectable “Prescribed Health Check” option. For example, user interface element 222 of patient computing device 200 may receive a user touch input at first user interaction element 812 indicating a selection of the Self Check option. Additionally, or alternatively, user interface element 222 of patient computing device 200 may receive a user touch input at second user interaction element 814 indicating a selection of the Prescribed Health Check option. In some examples, based on a user selection of one or both of the Self Check option or the Prescribed Health Check option, patient application 208 may proceed to one or more other interactive pages. Additionally, or alternatively, based on a user selection of one or both of the Self Check option or the Prescribed Health Check option, patient application 208 may update first test select interactive page 800A to show the selected option (e.g., color the selected option a different shade). In some examples, the self check is different that the prescribed health check in that the self check is performed at the request of the patient, whereas the prescribed health checks are performed according to a schedule set by a physician or other clinician.

Third user interaction element 816 represents a “Prescribed Nonstress Test” option. In some examples, a nonstress test is different test than the self check and the prescribed health check. In some examples, user interface element 222 of patient computing device 200 may receive a user touch input at third user interaction element 816 indicating a selection of the Prescribed Nonstress Test option. In some examples, there may be a fourth user interaction element (not illustrated in FIG. 8A) that includes a “self check” option for a nonstress test. The self check option for the nonstress test represents a patient-elected stress test, whereas the prescribed nonstress of third user interaction element 816 is performed according to a schedule set by a clinician.

Clock visual element 822 may indicate that prescribed health checks are to be performed according to a schedule, and deadline visual element 824 may indicate a date and/or a time at which the next prescribed health check is due. For example, deadline visual element 824 instructs the patient to perform a prescribed health check by Aug. 31, 2022. Although deadline visual element 824 does not indicate a time of day, other example deadline visual elements may include a time of day.

FIG. 8B is a conceptual diagram illustrating a second test select interactive page 800B, in accordance with one or more techniques of this disclosure. As seen in FIG. 8B, test select interactive page 800B includes interaction elements 814, 816. Test select interactive page 800B further includes a clock visual element 822 and a deadline visual element 824. In some examples, test select interactive page 800B may be substantially the same as test select interactive page 800A of FIG. 1 except that test select interactive page 800B does not include a user interactive element for a “self check.”

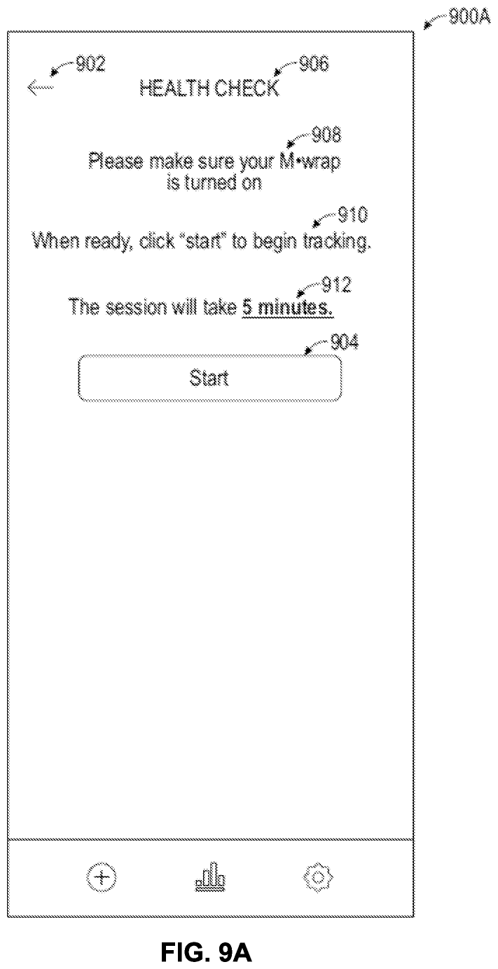

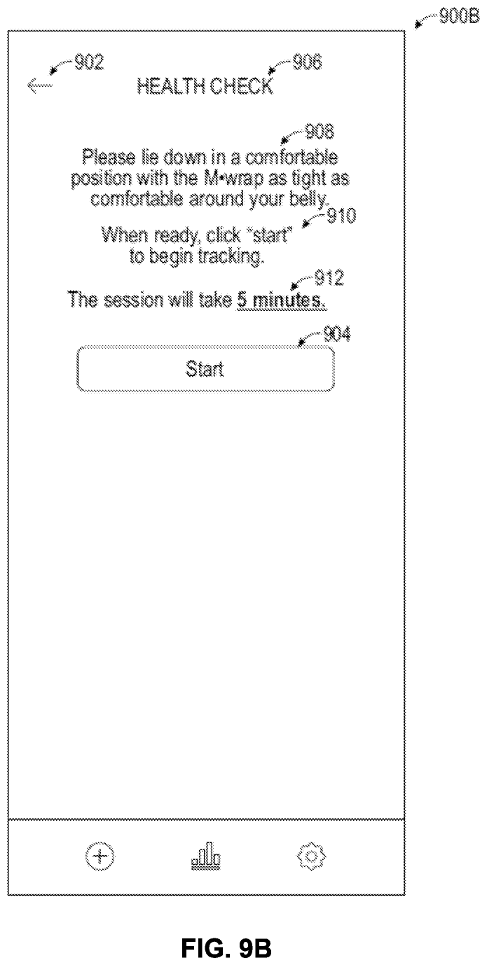

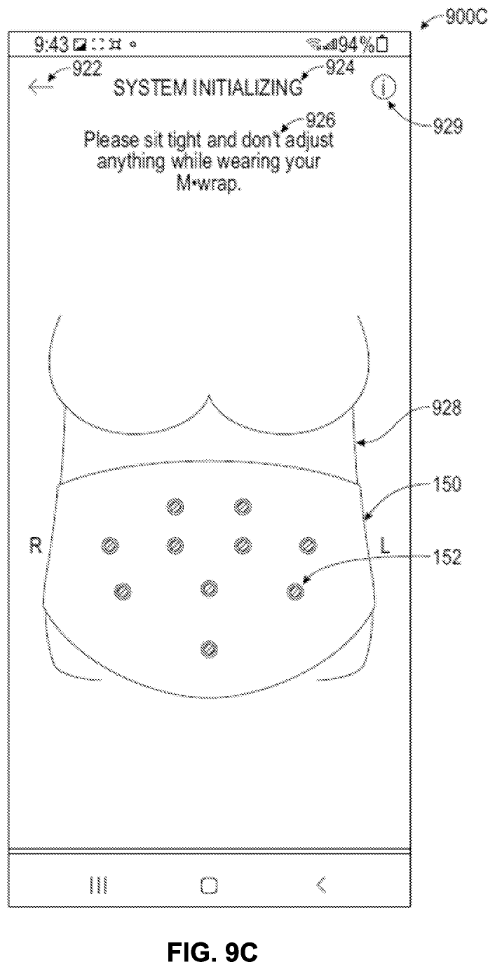

FIG. 9A is a conceptual diagram illustrating a first health check information interactive page 900A, in accordance with one or more techniques of this disclosure. As seen in FIG. 9A, health check information interactive page 900A includes a first user interaction element 902 and a second user interaction element 904. The health check information interactive page 900A also includes a first visual element 906, a second visual element 908, a third visual element 910, and a fourth visual element 912.

Computing device 200 may, in some examples, perform a test to make sure that a connection between computing device 200 and wearable device 150. If the connection between computing device 200 and wearable device 150 is insufficient, computing device 200 may output a message to first health check information interactive page 900A that the connection is insufficient.

In some examples, user interface element 222 of patient computing device 200 may display the health check information interactive page 900A in response to receiving a user selection of a self check option or a prescribed health check option on another interactive page (e.g., interactive page 800A or interactive page 800B). First user interaction element 902 may represent a “back arrow” that, when selected, will cause user interface element 222 of patient computing device 200 to display a previous interactive page (e.g., interactive page 800A or interactive page 800B). Second user interaction element 904 may represent a “start button.” When a user presses the start button, patient application 208 may proceed to one or more interactive pages relating to the performance of a health check.

First visual element 906 indicates that health check information interactive page 900A provides information corresponding to a “HEALTH CHECK.” Second visual element 908 informs the patient to make sure that wearable device 150 is turned on. Third visual element 910 informs the patient to select second user interaction element 904 when the patient is ready to start the health check. Fourth visual element 912 informs the patient that the health check will take 5 minutes. Fourth visual element 912 is not limited to indicating five minutes. Fourth visual element 912 may indicate any amount of time. In some examples, a clinician may program one or more other messages into health check information interactive page 900A that provide additional instructions and/or information relating to the health check.

FIG. 9B is a conceptual diagram illustrating a second health check information interactive page 900B, in accordance with one or more techniques of this disclosure. As seen in FIG. 9B, health check information interactive page 900B includes a first user interaction element 902 and a second user interaction element 904. The health check information interactive page 900A also includes a first visual element 906, a second visual element 908, a third visual element 910, and a fourth visual element 912. Health check information interactive page 900B may be substantially the same as health check information interactive page 900A of FIG. 9A except that second visual element 908 instructs the patient to lie down in a comfortable position instead of instructing the patient to make sure that the wearable device 150 is turned on.

Computing device 200 may, in some examples, perform a test to make sure that a connection between computing device 200 and wearable device 150. If the connection between computing device 200 and wearable device 150 is insufficient, computing device 200 may output a message to second health check information interactive page 900B that the connection is insufficient.

FIG. 9C is a conceptual diagram illustrating a health check initialization interactive page 900C, in accordance with one or more techniques of this disclosure. As seen in FIG. 9C, health check initialization interactive page 900C includes a first user interaction element 922, a first visual element 924, a second visual element 926, a third visual element 928, and a second user interaction element 929. In some examples, computing device 200 may display health check initialization interactive page 900C in response to a user selection of second user interaction element 904 of first health check information interactive page 900A and/or second health check information interactive page 900B. In other words, health check initialization interactive page 900C may represent the interactive page displayed by computing device 200 in response to user input to start a health check.

In some examples, first user interaction element 922 may represent a back arrow that, when selected, causes computing device 200 to display a previous interactive page. For example, when first user interaction element 922 is selected, computing device 200 may display first health check information interactive page 900A and/or second health check information interactive page 900B. In other examples, when first user interaction element 922 is selected, computing device 200 may display first test select interactive page 800A and/or second test select interactive page 800B. First visual element 924 may include the text “SYSTEM INITIALIZING.” Second visual element 926 may instruct the patient to wait without adjusting the wearable device 150. Third visual element 928 may include an avatar of a patient wearing wearable device 150 including sensors 152.

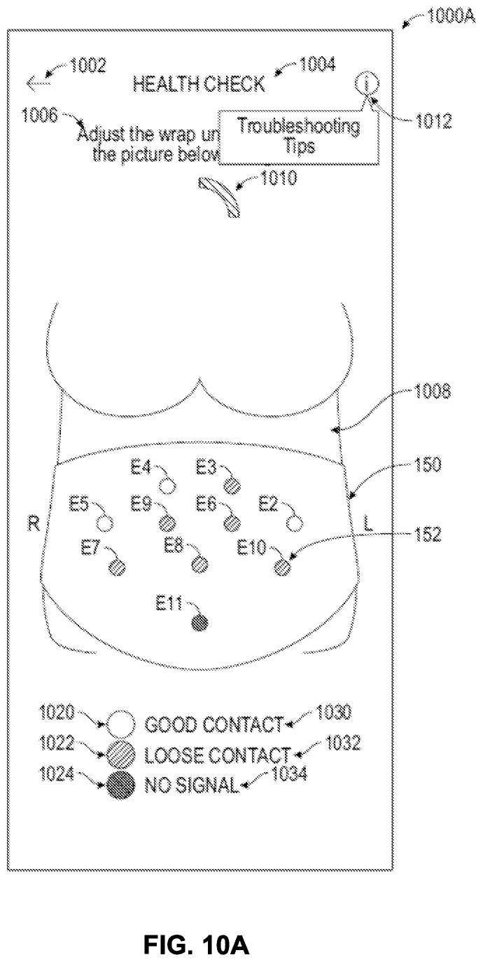

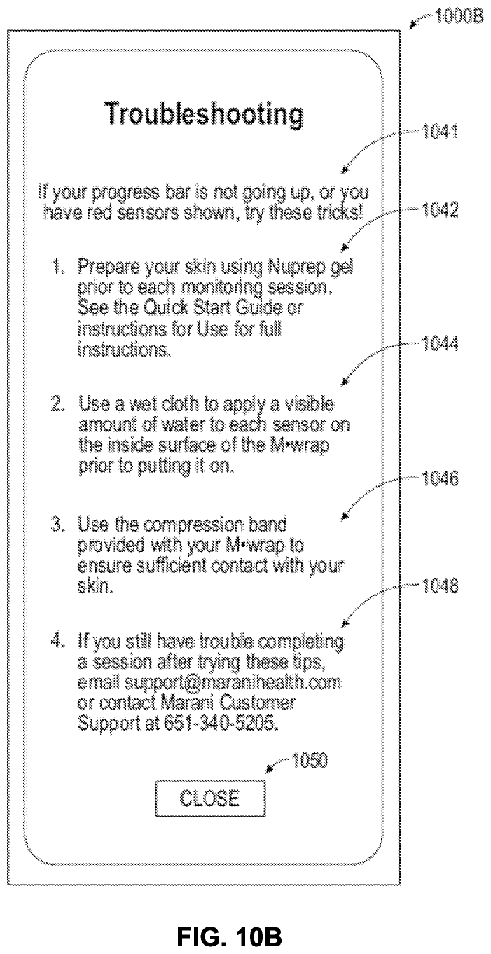

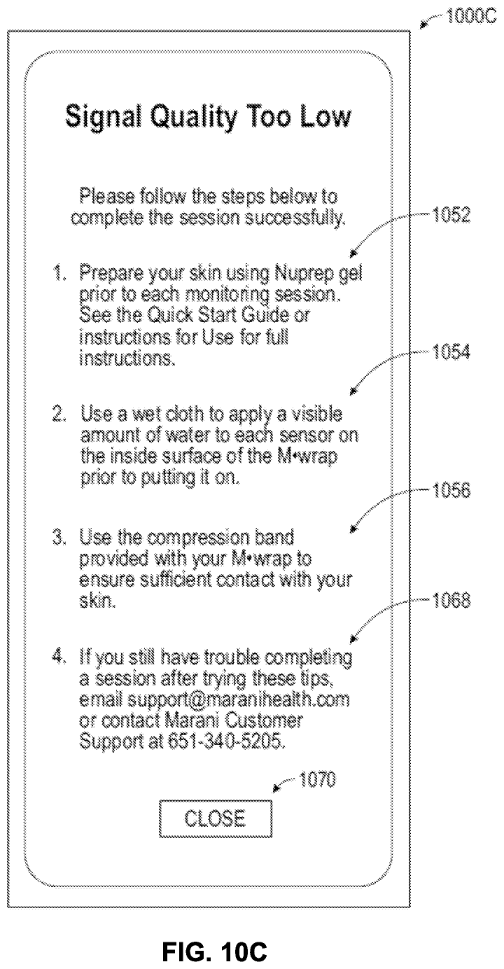

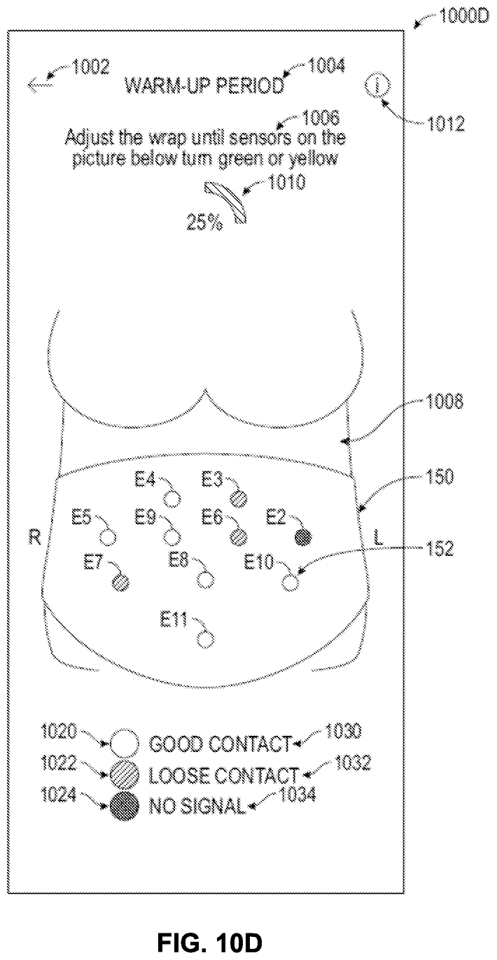

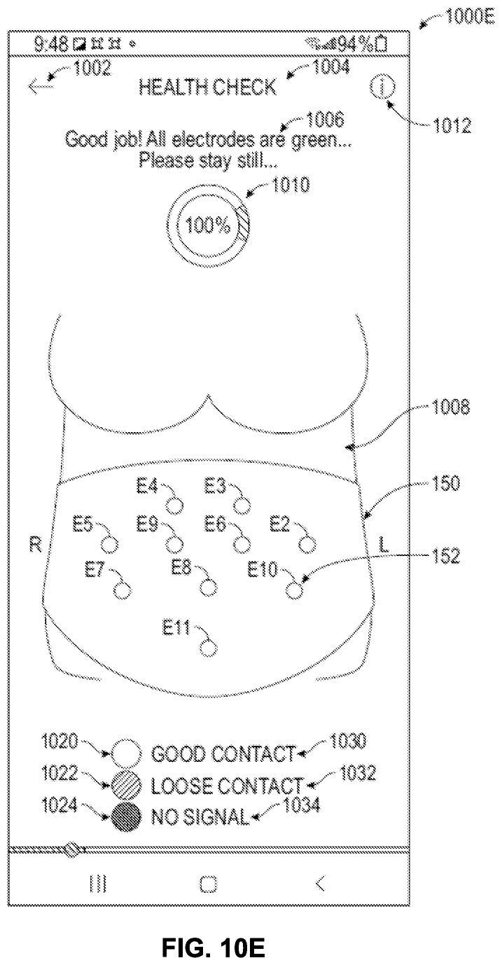

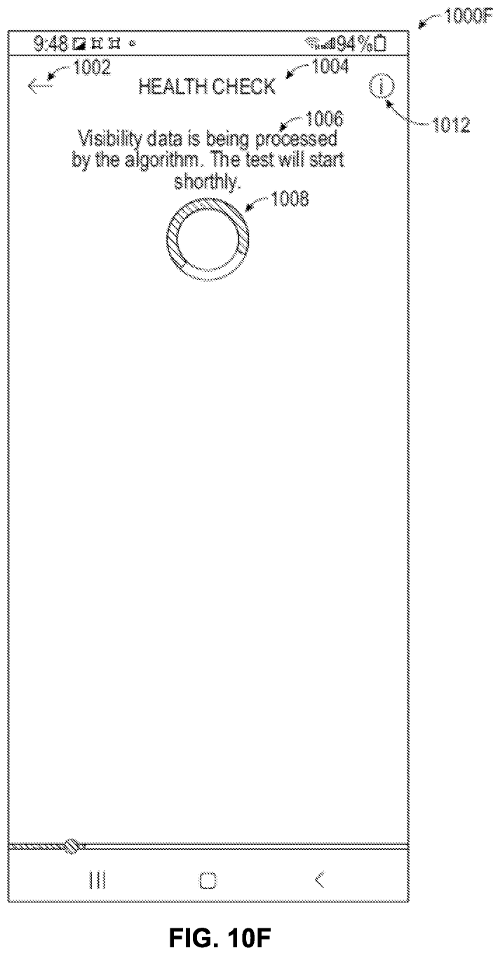

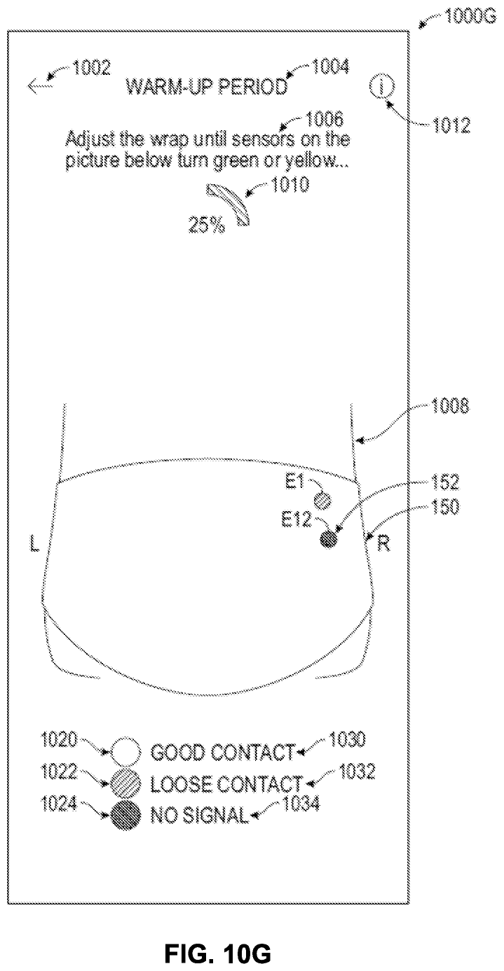

FIG. 10A is a conceptual diagram illustrating a first health check sensor contact interactive page 1000A, in accordance with one or more techniques of this disclosure. As seen in FIG. 10A, health check sensor contact interactive page 1000A includes a first user interaction element 1002, a first visual element 1004, a second visual element 1006, a third visual element 1008, a third visual element 1010 and a second user interaction element 1012. FIG. 10A also includes a legend having a first indicator 1020, a second indicator 1022, a third indicator 1024, and a first descriptor 1030, a second descriptor 1032, and a third descriptor 1034.

In some examples, user interface element 222 of patient computing device 200 may display the health check sensor contact interactive page 1000A in response to a user selection of the second user interaction element 904 of health check information interactive page 704, but this is not required. User interface element 222 may display the health check sensor contact interactive page 1000A at any time in the process. First user interaction element 1002 may represent a back arrow that, when selected, will cause user interface element 222 of patient computing device 200 to display a previous interactive page (e.g., interactive page 900A or interactive page 900B). First visual element 1004 indicates that health check sensor contact interactive page 1000A provides information corresponding to a “HEALTH CHECK.” In some examples, first visual element 1004 may include text other than “HEALTH CHECK” indicating that interactive page 1000A corresponds to a sensor contact check process. For example, first visual element 1004 may include the text “WARM-UP PERIOD.” Second visual element 1006 may include instructions for a user to complete at the corresponding stage in the health check.

As seen in FIG. 10A, health check sensor contact interactive page 1000A includes a third visual element 1008 that resembles an avatar of a patient. The patient avatar includes wearable device 150 and sensors 152. Each sensor of sensors 152 is shaded according to a quality of the contact between the respective sensor and the patient. For example, the legend of FIG. 10A shows that sensors shaded according to the color of first indicator 1020 have “GOOD CONTACT” with the patient as described by first descriptor 1030, sensors shaded according to the color of second indicator 1022 have “LOOSE CONTACT” with the patient as described by second descriptor 1032, and sensors shaded according to the color of third indicator 1024 have “NO SIGNAL” as described by third descriptor 1034. In some examples, “good contact” corresponds to a high quality of contact between the respective sensor and the patient, “loose contact” corresponds to a low quality of contact between the respective sensor and the patient, and “no signal” corresponds to no contact between the respective sensor and the patient. In some examples, second visual element 1006 includes one or more instructions for the patient to adjust the wearable device 150 until each of sensors 152 have “GOOD CONTACT” with the patient. Fourth visual element 1010 may represent a progress wheel that indicates how much progress has been made in achieving good contact between sensors 152 and the patient.

Sensors 152 may include sensors E2-E11 of FIG. 4. In some examples, sensors 152 may include one or more additional sensors that are not displayed on health check sensor contact interactive page 1000A. For example, sensors 152 may include sensors E1 and E12 of FIG. 4 located on a back side of wearable device 150. A front side of wearable device 150 is shown in FIG. 4. In some examples, computing device 200 may be configured to display another separate interactive page that indicates a level of contact between sensors E1 and E12 and the patient. In some examples, computing device 200 may be configured to indicate the level of contact between sensors E1 and E12 and the patient on the same interactive page 1000A that indicates the level of contact between sensors E2-E11 and the patient. In some examples, computing device 200 may cause health check sensor contact interactive page 1000A to display the level of contact between sensors E1 and E12 using one or more visual elements to the side of the visual element 1008 resembling the avatar of the patient. As seen in FIG. 10A, interactive page 1000A indicates that sensors E2, E4, and E5 have good contact with the patient, sensors E3, E6, E7, E8, E9, and E10 have loose contact with the patient, and sensor E11 has no contact with the patient.

In some examples, health check sensor contact interactive page 1000A includes a second user interaction element 1012 that redirects to a list of troubleshooting tips for achieving good contact between each sensor of sensors 152. For example, when computing device 200 receives a user selection of second user interaction element 1012 while health check sensor contact interactive page 1000A is displayed, computing device 200 may display another interactive page that provides one or more suggestions or “tips” for achieving good contact between each sensor of sensors 152.

Health check sensor contact interactive page 1000A may update the status of sensors 152 in real-time as the patient views user interface element 222 so that the patient may adjust wearable device 150 until the sensors have good contact with the patient. This feature may improve a quality of data from the health check as compared with systems that do not inform the patient of sensor contact status. For example, if patient adjusts wearable device 150 to improve the contact of sensor E11 with the patient, computing device 200 may transition the icon corresponding to electrode E11 to indicate that electrode E11 has good contact with the patient (e.g., update the icon corresponding to electrode E11 to have the first indicator 1020 instead of the third indicator 1024).

In some examples, the “level of contact” of each sensor 152 indicated on any of the interactive pages shown and described herein, such as interactive page 1000A of FIG. 10A, is determined based on the quality of the biopotential signal(s) detected by the respective sensor 152. In general, the icons indicative of the level of contact of each sensor provide feedback to the user to help to ensure that the system is able to acquire biopotential signals of sufficient quality (e.g., signals that do not contain excessive noise) that can be reliably used for purposes of predicting one or more maternal and/or fetal health outcomes. For example, the biopotential signal(s) detected by each of sensors 152 may be analyzed, either alone or in various combinations, to determine one or more signal quality metrics indicative of an amount of noise contained in the signal(s), and thus the level of contact between the respective sensor and the patient. The analysis may be performed by patient computing device 200 and/or FMS 300. Examples of signal quality metrics include, but are not limited to, any one or more of a signal-to-noise ratio (SNR), a noise-to-noise ratio, an error rate or ratio, a signal strength and/or power, and/or any other metric for measuring signal quality. The signal quality metrics may also include one or more metrics based on the timing or morphology of the acquired biopotential signals or events contained within the biopotential signals, such as cardiac, respiratory, or movement events.

In some examples, computing system 200 is configured to determine that each sensor of the set of sensors corresponding to a signal quality metric that satisfies a first noise ratio threshold has good contact with the patient. In some examples, computing system 200 is configured to determine that each sensor of the set of sensors 152 corresponding to a signal quality metric within a range from a second noise ratio threshold to the first noise ratio signal has loose contact with the patient. In some examples, computing system 200 is configured to determine that each sensor of the set of sensors 152 corresponding to a signal quality metric that satisfies a second noise ratio threshold has loose contact with the patient. In some examples, the system continuously monitors the signal quality of the biopotential signals acquired by sensors 152 throughout a monitoring session. In this way, the system may dynamically adjust which acquired biopotential signals are used for purposes of analysis and prediction based on the signal quality metrics. In addition, if at any time during a monitoring process the number of sensors for which the signal quality is determined to be insufficient (e.g., “loose” or “no contact”) drops below a predetermined number as described herein, the system may display an interactive page including suggestions for improving the level of contact between the sensors 152 and the patient.

FIG. 10B is a conceptual diagram illustrating a troubleshooting interactive page 1000B that is accessible via the health check sensor contact interactive page 1000A of FIG. 10A, in accordance with one or more techniques of this disclosure. As seen in FIG. 10B, troubleshooting interactive page 1000B includes a message 1041, a first suggestion 1042, a second suggestion 1044, a third suggestion 1046, and a fourth suggestion 1048. In some examples, computing device 200 displays troubleshooting interactive page 1000B in response to receiving a user selection of second user interaction element 1012 of the health check sensor contact interactive page 1000A of FIG. 10A. The message 1041 of troubleshooting interactive page 1000B may instruct the patient to follow suggestions on the troubleshooting interactive page 1000B if the health check sensor contact process is not resulting in sufficient contact between sensors 152 and the patient.

First suggestion 1042 instructs the patient to prepare their skin using a gel prior to each monitoring session. In some examples, gel applied to the skin may improve contact between sensors 152 and the patient as compared with not applying gel to the skin. This means that it may be beneficial for troubleshooting interactive page 1000B to instruct the patient to apply gel to the skin prior to each monitoring session if the patient is not able to achieve sufficient sensor contact, because if the patient forgot to apply gel this suggestion may help the patient to remember to apply the gel.

Second suggestion 1044 may instruct the patient to use a wet cloth to apply a visible amount of water to each of sensors 152 on the inside surface of the wearable device 150 prior to putting on the wearable device. Dry skin may, in some cases, prevent sensors 152 from achieving good contact with the patient. In some examples, excessively wet skin also prevents sensors 152 from achieving good contact with the patient. Therefore, second suggestion 1044 instructs the patient to apply a visible amount of water to each sensor of sensors 152 so that there is some, but not too much moisture between each sensor and the patient.

Third suggestion 1046 may instruct the patient to use a compression band of the wearable device 150 to ensure sufficient contact with the skin. In some examples, the compression of wearable device 150 may be configured to tighten or loosen the wearable device 150 on the patient's body. In some examples, tightening the compression band may improve a contact of sensors 152 with the patient. It may be beneficial to include third suggestion 1046 on the troubleshooting interactive page 1000B so that patient remembers to try tightening the compression band if the sensor contact is insufficient.

Fourth suggestion 1048 of troubleshooting interactive page 1000B tells the patient to contact a manufacturer of wearable device 150 if the patient is unable to achieve strong contact between sensors 152 and the patient. The manufacturer of wearable device 150 may have a customer service division for assisting patients with using the wearable device 150. Fourth suggestion 1048 may provide an email address and/or a telephone number for customer service. This may allow the patient to receive further assistance if none of the other suggestions help the patient to achieve good contact between sensors 152 and the patient.

The troubleshooting interactive page 1000B is not limited to the suggestions illustrated in FIG. 10B. One or more other example troubleshooting interactive pages may include suggestions in addition to or alternatively to the suggestions illustrated in FIG. 10B. For example, a troubleshooting interactive page may include a suggestion that warns the patient against using consumer creams or lotions before using the wearable device 150. Although some gels may improve sensor contact, consumer creams and lotions may prevent sensors 152 of wearable device 150 from achieving good contact with the patient. This means that a presence of creams and lotions on the patient may be the cause loose contact or no contact between sensors 152 and the patient.

Another example suggestion may remind the patient to ensure that each of sensors 152 are in direct contact with the patient's skin. When the wearable device 150 is not worn properly, this may cause one or more sensors of sensors 152 to not be in direct contact with the patient's skin. By including a suggestion on the troubleshooting interactive page reminding the patient to check contact, computing device 200 may help the patient to adjust the wearable device 150 so that a greater number of sensors 152 are in direct contact with the patient's skin, and good contact is achieved.

Troubleshooting interactive page 1000B is not limited to the example suggestions 1042-1048 illustrated in FIG. 10B and/or the other example suggestions described herein. In some examples, troubleshooting interactive page 1000B may include one or more suggestions in addition to or alternatively to the suggestions 1042-1048 illustrated in FIG. 10B and the suggestions described here. Troubleshooting interactive page 1000B may include a user interaction element 1050 for closing troubleshooting interactive page 1000B and reverting to interactive page 1000A.

FIG. 10C is a conceptual diagram illustrating an error interactive page 1000C, in accordance with one or more techniques of this disclosure. As seen in FIG. 10C, error interactive page 1000C includes a first message 1052, a second message 1054, a third message 1056, a fourth message 1058, and a fifth message 1060. In some examples, computing device 200 displays error interactive page 1000C in response to the number of sensors 152 having good contact with the patient not exceeding a threshold number of sensors after a predetermined amount of time (e.g., 5 minutes) following a start of the sensor contact process. In some examples, the “sensor contact process” refers to the process initiated by a user input to user interaction element 904 of FIG. 9A and/or FIG. 9B.

First message 1052 instructs the patient to prepare their skin using a gel prior to each monitoring session. In some examples, gel applied to the skin may improve contact between sensors 152 and the patient as compared with not applying gel to the skin. This means that it may be beneficial for troubleshooting interactive page 1000B to instruct the patient to apply gel to the skin prior to each monitoring session if the patient is not able to achieve sufficient sensor contact, because if the patient forgot to apply gel this message may help the patient to remember to apply the gel.

Second message 1054 may instruct the patient to use a wet cloth to apply a visible amount of water to each of sensors 152 on the inside surface of the wearable device 150 prior to putting on the wearable device. Dry skin may, in some cases, prevent sensors 152 from achieving good contact with the patient. In some examples, excessively wet skin also prevents sensors 152 from achieving good contact with the patient. Therefore, second message 1054 instructs the patient to apply a visible amount of water to each sensor of sensors 152 so that there is some, but not too much moisture between each sensor and the patient.

Third message 1056 may instruct the patient to use a compression band of the wearable device 150 to ensure sufficient contact with the skin. In some examples, the compression of wearable device 150 may be configured to tighten or loosen the wearable device 150 on the patient's body. In some examples, tightening the compression band may improve a contact of sensors 152 with the patient. It may be beneficial to include third message 1056 on the troubleshooting interactive page 1000B so that patient remembers to try tightening the compression band if the sensor contact is insufficient.

Fourth message 1058 of error interactive page 1000C tells the patient to contact a manufacturer of wearable device 150 if the patient is unable to achieve strong contact between sensors 152 and the patient. The manufacturer of wearable device 150 may have a customer service division for assisting patients with using the wearable device 150. Fourth message 1058 may provide an email address and/or a telephone number for customer service. This may allow the patient to receive further assistance if none of the other suggestions help the patient to achieve good contact between sensors 152 and the patient. Error interactive page 1000C is not limited to the example suggestions 1052-1058 illustrated in FIG. 10C and/or the other example suggestions described herein. In some examples, error interactive page 1000C may include one or more suggestions in addition to or alternatively to the suggestions 1052-1058 illustrated in FIG. 10C and the suggestions described here. Error interactive page 1000C may include a user interaction element 1070 for closing error interactive page 1000C and reverting to interactive page 1000A, or another interactive page.

FIG. 10D is a conceptual diagram illustrating a second health check sensor contact interactive page 1000D, in accordance with one or more techniques of this disclosure. As seen in FIG. 10D, second health check sensor contact interactive page 1000D includes a first user interaction element 1002, a first visual element 1004, a second visual element 1006, a third visual element 1008, a fourth visual element 1010, and a second user interaction element 1012. FIG. 10D also includes a legend having a first indicator 1020, a second indicator 1022, a third indicator 1024, and a first descriptor 1030, a second descriptor 1032, and a third descriptor 1034.

In some examples, interactive page 1000D is substantially the same as interactive page 1000A except that first visual element 1004 includes the text “WARM-UP PERIOD” instead of the text “HEALTH CHECK,” and fourth visual element 1010 indicates that the sensor contact process is 25% complete, and at least some of sensors E2-E11 are associated with different descriptors. For example, in FIG. 10D, senor E2 has “NO SIGNAL,” sensors E3, E6, and E7 have “LOOSE CONTACT” with the patient, and sensors E4, E5, E8, E9, E10, and E11 have “GOOD CONTACT” with the patient. In some examples, as any one or combination of sensors E2, E3, E6, and E7 improve their contact with the patient, the progress wheel of fourth visual element 1010 may increase from 25% to a higher percentage value. When sufficient contact between sensors 152 and the patient is achieved, the progress wheel of fourth visual element 1010 may reach 100% and the sensor contact process may be complete. In some examples, the icons on interactive page 1000D may update in real time based on the contact between the sensors and the patient improving and/or worsening.

FIG. 10E is a conceptual diagram illustrating a third health check sensor contact interactive page 1000E, in accordance with one or more techniques of this disclosure. As seen in FIG. 10E, third health check sensor contact interactive page 1000E includes a first user interaction element 1002, a first visual element 1004, a second visual element 1006, a third visual element 1008, a fourth visual element 1010, and a second user interaction element 1012. FIG. 10E also includes a legend having a first indicator 1020, a second indicator 1022, a third indicator 1024, and a first descriptor 1030, a second descriptor 1032, and a third descriptor 1034. In some examples, interactive page 1000E is substantially the same as interactive page 1000A except that second visual element 1006 in FIG. 10E indicates that all of sensors 152 have good contact with the patient, whereas second visual element 1006 of FIG. 10A indicates that wearable device 150 needs to be adjusted to improve contacts. Furthermore, visual element 1008 indicates that each of electrodes E2-E11 are associated with the first indicator 1020 that is associated with good contact. Health check sensor contact interactive page 1000E also includes a progress indicator 1012 that indicates that a fitting of the wearable device 150 is complete (e.g., 100%).

In some examples, computing device 200 may transition from displaying interactive page 1000A and/or interactive page 1000D to displaying interactive page 1000E when a sufficient number of sensors have good contact with the patient. A sufficient number of sensors may, in some cases, be greater than or equal to a threshold number of sensors that have good contact with the skin of the patient. In some examples, the threshold number of sensors may represent the total number of sensors 152. In some examples, the threshold number of sensors may be less than the total number of sensors 152. In some examples, the threshold number of sensors is five sensors. In any case, when greater than or equal to the threshold number of sensors of sensors 152 have a good level of contact with the patient, computing device 200 may transition from displaying interactive page 1000A to displaying interactive page 1000E, indicating to the patient that the wearable device 150 is fitted and the health check is ready to proceed.

In some examples, computing device 200 may display interactive page 1000E without first displaying interactive page 1000A and/or interactive page 1000D if greater than or equal to the threshold number of sensors have good contact with the patient when computing device 200 receives a user input to second user interaction element 904 of interactive page 900A and/or interactive page 900B. When greater than or equal to the threshold number of sensors have good contact with the patient, it may be unnecessary for the patient to adjust wearable device 150, obviating a need to display interactive page 1000A and/or interactive page 1000D.

FIG. 10F is a conceptual diagram illustrating an example of a health check visibility interactive page 1000F, in accordance with one or more techniques of this disclosure. As seen in FIG. 10F, health check visibility interactive page 1000F includes a first user interaction element 1002, a first visual element 1004, a second visual element 1006, a third visual element 1008, and a second user interaction element 1012. First user interaction element 1002 may represent a back arrow that, when selected, will cause user interface element 222 of patient computing device 200 to display a previous interactive page (e.g., interactive page 1000E). For example, user interface element 222 may display health check visibility interactive page 1000F after displaying interactive page 1000E after displaying interactive page 1000E which indicates that sensors 152 have good contact with the patient.

In some examples, health check visibility interactive page 1000F may correspond to a yield test performed by computing device 200 and/or FMS 300. As described herein, a “yield test” may represent a test of signals collected by wearable device 150 via sensors 152. During a yield test, computing device 200 and/or FMS 300 may evaluate a quality, or “yield,” of one or more signals initially collected by wearable device 150 via sensors 152 after sensors 152 achieve good contact with the patient. If the yield test indicates that the quality of the one or more signals initially collected by wearable device 150 via sensors 152 does not meet a quality threshold, computing device 200 may display error interactive page 1000C of FIG. 10C. If the yield test indicates that the quality of the one or more signals initially collected by wearable device 150 via sensors 152 does meet a quality threshold, computing device 200 may proceed with a full health check. In some examples, a yield test measures a percentage of time that the collected signals yield data sufficient for measuring maternal and/or fetal cardiac parameters (e.g., maternal and/or fetal heart rate).

FIG. 10G is a conceptual diagram illustrating a fourth health check sensor contact interactive page 1000G, in accordance with one or more techniques of this disclosure. As seen in FIG. 10G, fourth health check sensor contact interactive page 1000G includes a first user interaction element 1002, a first visual element 1004, a second visual element 1006, a third visual element 1008, a fourth visual element 1010, and a second user interaction element 1012. FIG. 10G also includes a legend having a first indicator 1020, a second indicator 1022, a third indicator 1024, and a first descriptor 1030, a second descriptor 1032, and a third descriptor 1034.

In some examples, interactive page 1000G is substantially the same as interactive page 1000D of FIG. 10D except that third visual element 1008 of interactive page 1000G is an avatar of the patient wearing the wearable device 150 from a view looking at a posterior side of the patient, whereas the third visual element 1008 of interactive page 1000D is an avatar of the patient wearing the wearable device 150 from a view looking at a anterior side of the patient. In some examples, the posterior side of wearable device 150 includes sensors E1 and E12, whereas the posterior side of wearable device 150 includes sensors E1-E11. As seen in FIG. 10G, sensor E1 has loose contact with the patient and sensor E12 has no contact with the patient.

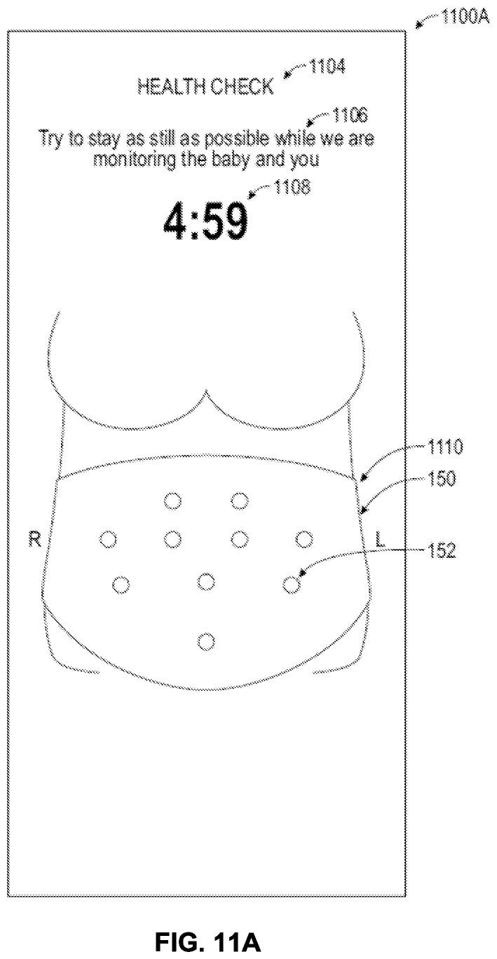

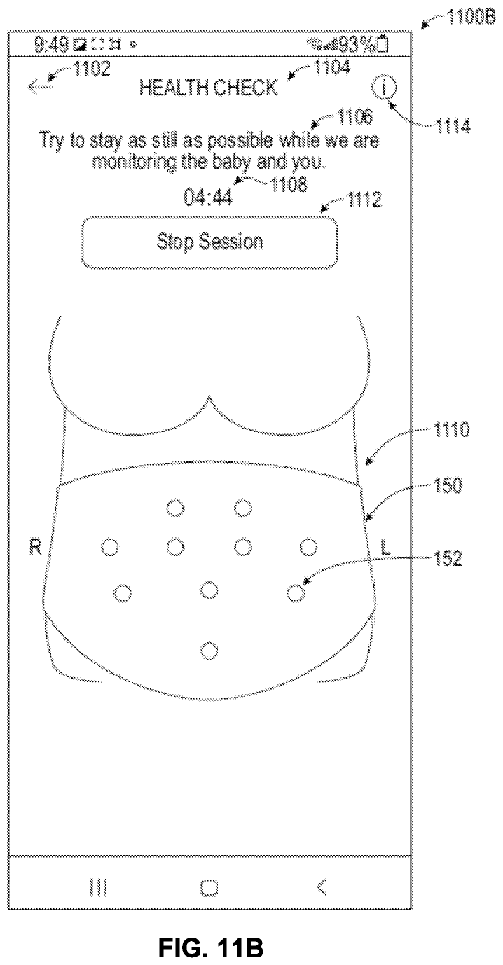

FIG. 11A is a conceptual diagram illustrating a first health check progress interactive page 1100A, in accordance with one or more techniques of this disclosure. As seen in FIG. 11A, health check progress interactive page 1100A includes a first visual element 1104, a second visual element 1106, a third visual element 1108, and a fourth visual element 1110. First visual element 1004 may include the words “HEALTH CHECK” to indicate that a health check process is currently in progress. Second visual element 1106 may include a message instructing the patient to remain as still as possible while the health check is in progress. Third visual element 1108 may include an amount of time that counts down to the end of the health check. For example, a health check may last for an amount of time (e.g., 5 minutes). Third visual element 1108 may count down so that the patient is informed as to how much longer the health check will last.

Fourth visual element 1110 resembles an avatar of a patient. The patient avatar includes wearable device 150 and sensors 152. In some examples, each sensor of sensors 152 is shaded according to a quality of the contact between the respective sensor and the patient. In the example of health check progress interactive page 1100A, each sensor of sensors 152 may have good contact with the patient.

In some examples, computing device 200 may display health check progress interactive page 1100A after computing device 200 and/or FMS 300 verifies that at least a threshold number of sensors 152 have good contact with the patient. In some examples, computing device 200 may display health check progress interactive page 1100A after computing device 200 and/or FMS 300 performs a yield test to evaluate signals collected by wearable device 150 via sensors 152. In some examples, computing device 200 and/or FMS 300 verifies that at least a threshold number of sensors 152 have good contact with the patient while computing device 200 displays interactive page 1000A and/or interactive page 100D. In some examples, computing device 200 and/or FMS 300 perform a yield test to evaluate signals collected by wearable device 150 via sensors 152 while computing device 200 displays interactive page 1000F. In some examples, computing device 200 and/or FMS 300 perform the health check when computing device 200 displays health check progress interactive page 1100A.

FIG. 11B is a conceptual diagram illustrating a second health check progress interactive page 1100B, in accordance with one or more techniques of this disclosure. As seen in FIG. 11B, health check progress interactive page 1100B includes a first user interaction element 1102, a first visual element 1104, a second visual element 1106, a third visual element 1108, a fourth visual element 1110, a second user interaction element 1112, and a third user interaction element 1114. In some examples, the health check progress interactive page 1100B is substantially the same as the health check progress interactive page 1100A of FIG. 11A, except that health check progress interactive page 1100B includes first user interaction element 1102, second user interaction element 1112, and third user interaction element 1114, and displays a different amount of time on third visual element 1108 (e.g., 04:44, as opposed to 04:59).

First user interaction element 1102 may represent a back arrow that, when selected, will cause user interface element 222 of patient computing device 200 to display a previous interactive page. Second user interaction element 1112 may represent a “stop session” button that, when selected, causes computing device 200 to terminate the health check. Third user interaction element 1114 may represent a troubleshooting button that, when selected, causes computing device 200 to display another screen including information corresponding to the health check.

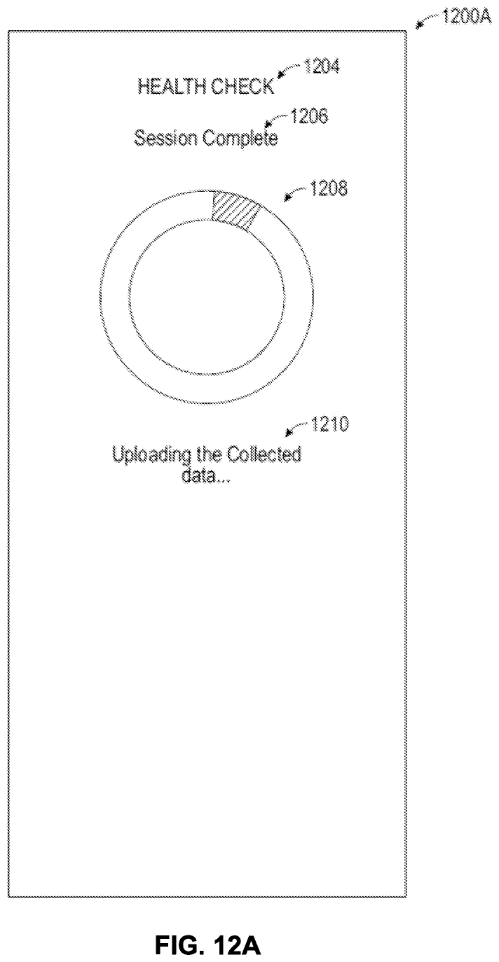

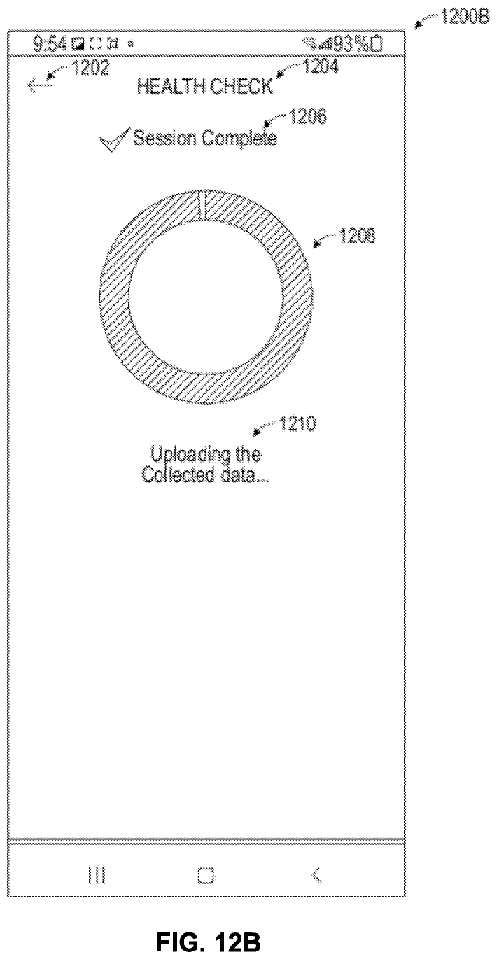

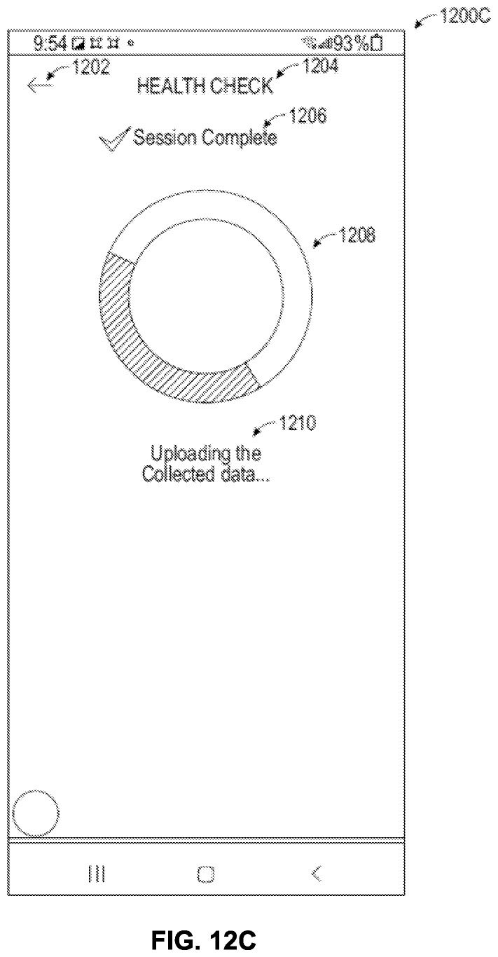

FIG. 12A is a conceptual diagram illustrating a first health check upload interactive page 1200A, in accordance with one or more techniques of this disclosure. As seen in FIG. 12A, first health check upload interactive page 1200A includes a first visual element 1204, a second visual element 1206, a third visual element 1208, and a fourth visual element 1210. First visual element 1204 may include the words “HEALTH CHECK” to indicate that health check data is currently being uploaded. Second visual element 1206 may include the words “SESSION COMPLETE” to indicate that computing device 200 has finished collecting data for the health check. Third visual element 1208 may include a progress wheel. In some examples, the progress wheel of third visual element 1208 may include a dark element and a light element. The dark element may expand around the progress wheel as the data is uploaded. Fourth visual element 1210 may include a message indicating that health check data is being uploaded. In some examples, computing device 200 may upload the health check data to FMS 300 for processing.

Computing device 200 may display health check upload interactive page 1200A after the health check is complete. For example, computing device 200 may display health check upload interactive page 1200A when third visual element 1108 of FIGS. 11A-11B counts down to zero and the health check is completed. That is, computing device 200 may transition from displaying interactive page 1100A and/or interactive page 1100B to displaying interactive page 1200A when the health check is complete, and data is ready for upload to FMS 300.

FIG. 12B is a conceptual diagram illustrating a second health check upload interactive page 1200B, in accordance with one or more techniques of this disclosure. As seen in FIG. 12B, second health check upload interactive page 1200B includes user interaction element 1202, a first visual element 1204, a second visual element 1206, a third visual element 1208, and a fourth visual element 1210. Second health check upload interactive page 1200B may be substantially the same as first health check upload interactive page 1200A of FIG. 12A except that second health check upload interactive page 1200B includes user interaction element 1202, and third visual element 1208 of FIG. 12B indicates a greater amount of upload process as compared with third visual element 1208 of FIG. 12A. For example, the dark portion of the progress wheel occupies a greater portion of the progress wheel in second health check upload interactive page 1200B as compared with the progress wheel of first health check upload interactive page 1200A. User interaction element 1202 may represent a back arrow that, when selected, will cause patient computing device 200 to display a previous interactive page on user interface element 222.

FIG. 12C is a conceptual diagram illustrating a third health check upload interactive page 1200C, in accordance with one or more techniques of this disclosure. As seen in FIG. 12C, third health check upload interactive page 1200C includes user interaction element 1202, a first visual element 1204, a second visual element 1206, a third visual element 1208, and a fourth visual element 1210. In some examples, the third health check upload interactive page 1200C is substantially the same as the second health check upload interactive page 1200B, except that third visual element 1208 represents a spinning dark portion that rotates clockwise or counterclockwise around third visual element 1208 without growing or shrinking in size.

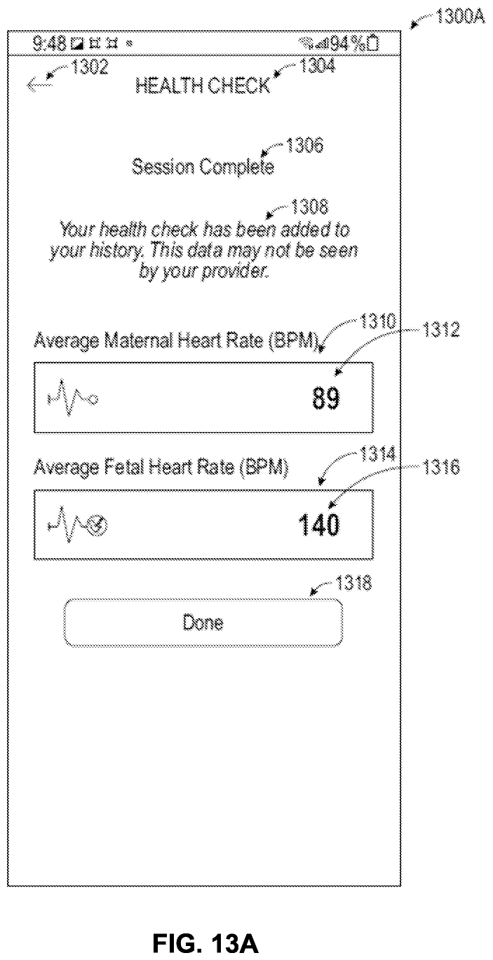

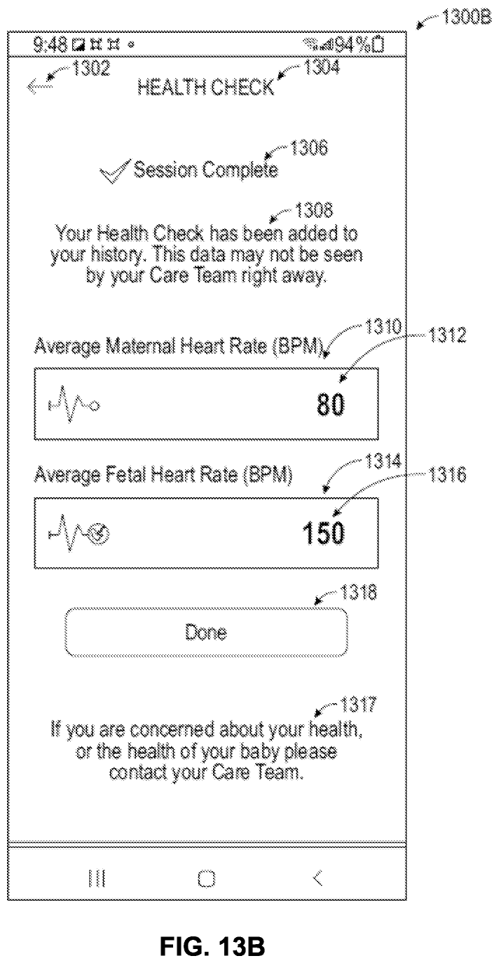

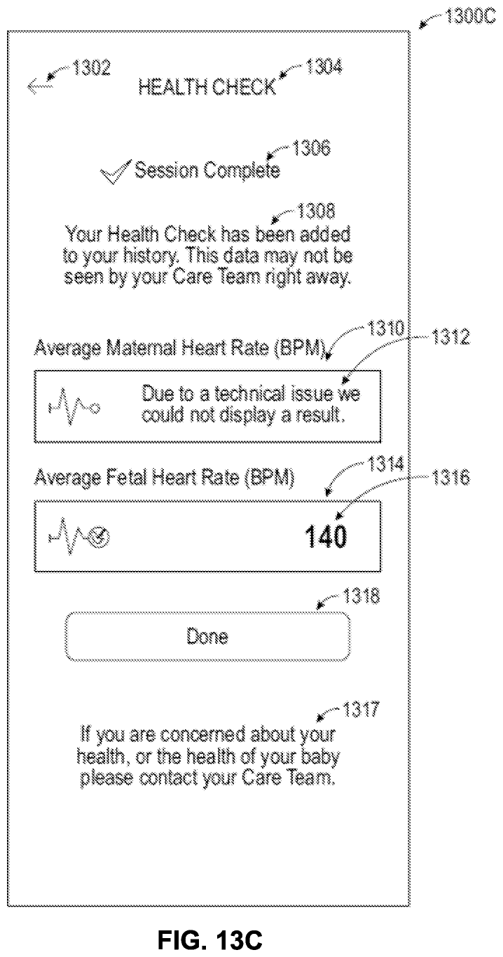

FIG. 13A is a conceptual diagram illustrating a first health check results interactive page 1300A, in accordance with one or more techniques of this disclosure. As seen in FIG. 13A, the first interactive page 1302A includes a first user interaction element 1302, a first visual element 1304, a second visual element 1306, a third visual element 1308, a fourth visual element 1310 including a first health check result 1312, a fifth visual element 1314 including a second health check result 1316. The first health check results interactive page 1300A also includes a second user interaction element 1318.

In some examples, computing device 200 may transition to displaying the first health check results interactive page 1300A after displaying any one or combination of the health check upload interactive pages 1200A-1200C. In some examples, computing device 200 may transition to displaying the first health check results interactive page 1300A after health check data is upload and health check results are received. User interaction element 1302 may represent a back arrow that, when selected, will cause patient computing device 200 to display a previous interactive page on user interface element 222. In some examples, the previous interactive page may represent any interaction page that precedes interactive page 1300A in the health check process.

First visual element 1304 may indicate that health check results interactive page 1300A indicates information corresponding to a health check. Second visual element 1306 may indicate that the health check session is complete. Third visual element 1308 includes a message that indicates that the health check has been added to the patient's medical history, and that the data corresponding to this health check is not visible to the patient' s provider. Fourth visual element 1310 includes data corresponding to average maternal heart rate, presented in beats per minute (BPM). Fourth visual element 1310 includes a first health check result 1312 indicating that the maternal heart rate corresponding to the health check is 89 BPM. Fifth visual element 1314 includes data corresponding to average fetal heart rate, presented in BPM. Fifth visual element 1314 includes a second health check result 1316 indicating that the fetal heart rate corresponding to the health check is 140 BPM. Second user interaction element 1318 may represent a “Done” button that, when pressed, indicates that the health check process is complete.

FIG. 13B is a conceptual diagram illustrating a second health check results interactive page 1300B, in accordance with one or more techniques of this disclosure. As seen in FIG. 13B, the second health check results interactive page 1300B includes a first user interaction element 1302, a first visual element 1304, a second visual element 1306, a third visual element 1308, a fourth visual element 1310 including a first health check result 1312, a fifth visual element 1314 including a second health check result 1316, and a sixth visual element 1317. The second health check results interactive page 1300B also includes a second user interaction element 1318.

In some examples, the second health check results interactive page 1300B is substantially the same as the first health check results interactive page 1300A except that third visual element 1308 of health check results interactive page 1300B includes a message indicating that “this data may not be seen by your Care Team right away” instead of “this data may not be seen by your provider” of FIG. 13A. Furthermore, the first health check result 1312 of FIG. 13B indicates a maternal heart rate of 80 BPM instead of 89 BPM indicated by the first health check result 1312 of FIG. 13A, and the second health check result 1316 of FIG. 13B indicates a maternal heart rate of 150 BPM instead of 140 BPM indicated by the second health check result 1316 of FIG. 13A. Health check results interactive page 1300B also includes a sixth visual element 1317 not present in interactive page 1300A of FIG. 13A which instructs that patient to contact the care team if the patient becomes concerned about a health of the patient or the baby.

FIG. 13C is a conceptual diagram illustrating a third health check results interactive page 1300C, in accordance with one or more techniques of this disclosure. As seen in FIG. 13B, the third health check results interactive page 1300C includes a first user interaction element 1302, a first visual element 1304, a second visual element 1306, a third visual element 1308, a fourth visual element 1310 including a first health check result 1312, a fifth visual element 1314 including a second health check result 1316, and a sixth visual element 1317. The second health check results interactive page 1300B also includes a second user interaction element 1318. In some examples, the third health check results interactive page 1300C is substantially the same as the second health check results interactive page 1300B except that first health check result 1312 of third health check results interactive page 1300C does not include a maternal heart rate value, but instead includes a message that the system could not display a result due to a technical issue. Additionally, second health check result 1314 of FIG. 13C includes a fetal heart rate of 140 BPM, whereas second health check result 1314 of FIG. 13B includes a fetal heart rate of 150 BPM.

In some examples, computing system 200 may perform a yield test based on the data collected during a health check (e.g., the data collected while computing system 200 displays interactive page 1100B and/or interactive page 1100A). If the yield test indicates that maternal cardiac data is insufficient to determine a reliable maternal heart rate, first health check result 1312 of third health check results interactive page 1300C may indicate that a maternal heart rate result could not be displayed due to a technical issue. In some examples, a health check results interactive page (not illustrated in FIGS. 13A-13C) may include a health check result indicating that fetal cardiac data is insufficient to determine a fetal maternal heart rate in response to a yield test revealing that fetal cardiac data is insufficient to determine a reliable fetal heart rate.

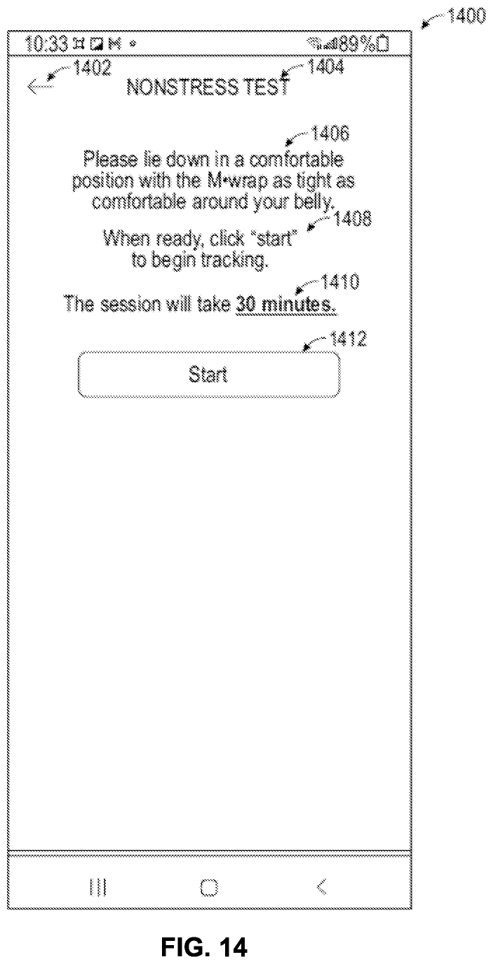

FIG. 14 is a conceptual diagram illustrating a nonstress test information interactive page 1400, in accordance with one or more techniques of this disclosure. As seen in FIG. 14, nonstress test information interactive page 1400 includes a first user interaction element 1402, a first visual element 1404, a second visual element 1406, a third visual element 1408, and a fourth visual element 1410. Nonstress test information interactive page 1400 includes a second user interaction element 1412.

Computing device 200 may, in some examples, perform a test to make sure computing device 200 and wearable device 150 are communicatively connected. For example, if communication device 200 and wearable device 150 are configured to communicate via a wireless Bluetooth connection, computing device 200 may check whether communication device 200 is paired with wearable device 150 and/or whether the wireless Bluetooth connection between communication device 200 and wearable device 150 is sufficient to support communication between the two devices. If the connection between computing device 200 and wearable device 150 is insufficient, computing device 200 may output a message to nonstress test information interactive page 1400 that the connection is insufficient.

Computing device 200 may, in some examples, display nonstress test information interactive page 1400 on user interface element 222 in response to a patient selection of a nonstress test from test select interactive page 800A and/or test select interactive page 800B of FIGS. 8A-8B. First user interaction element 1402 may represent a back arrow that, when selected, will cause user interface element 222 of patient computing device 200 to display a previous interactive page (e.g., interactive page 800A or interactive page 800B).

First visual element 1404 indicates that nonstress test information interactive page 1400 provides information corresponding to a “NONSTRESS TEST.” Second visual element 1406 informs the patient to lie down in a comfortable position with wearable device 150 wrapped as tight as possible without causing discomfort to the patient. Third visual element 1408 informs the patient to select second user interaction element 1412 when the patient is ready to start the nonstress test. Fourth visual element 1410 informs the patient that the nonstress test will take 30 minutes. The nonstress test is not limited to lasting 30 minutes. The nonstress test may last for an amount of time longer than or shorter than 30 minutes. In some examples, a clinician may program one or more other messages into nonstress test information interactive page 1400 that provide additional instructions and/or information relating to the nonstress test.

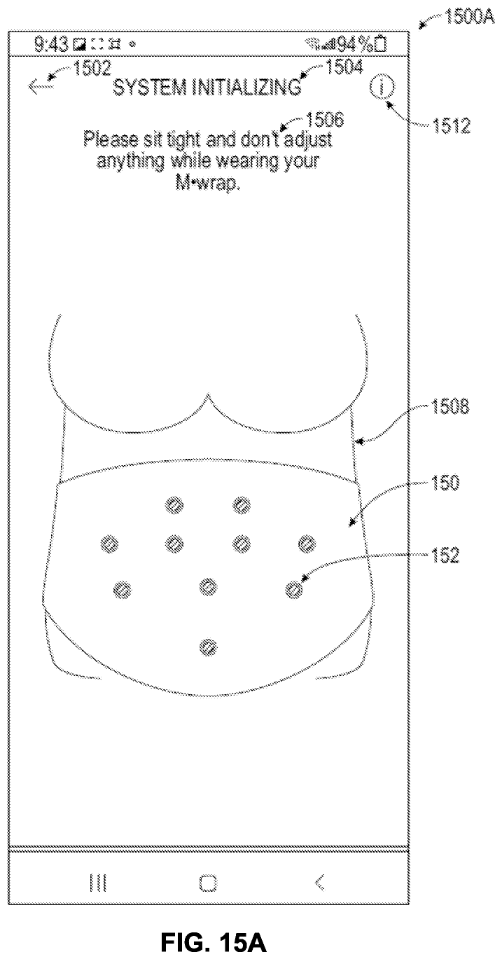

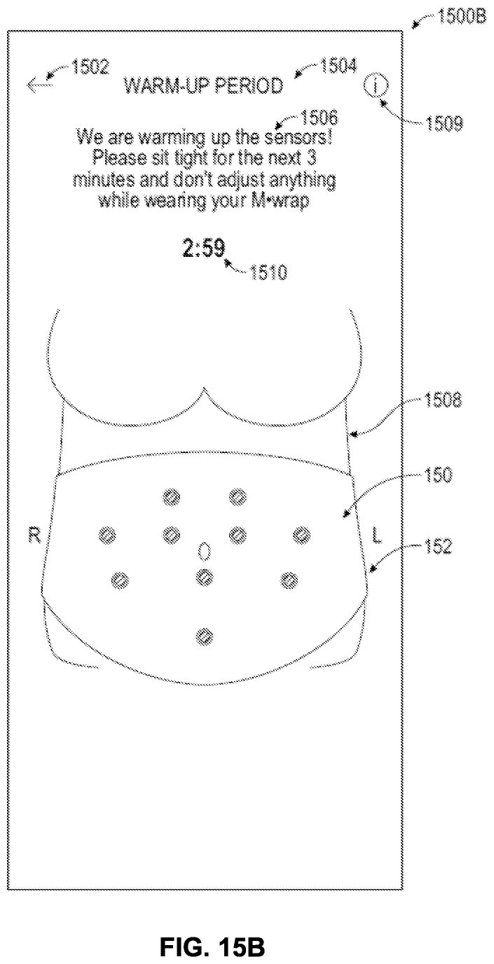

FIG. 15A is a conceptual diagram illustrating a first nonstress test initialization interactive page 1500A, in accordance with one or more techniques of this disclosure. As seen in FIG. 15A, nonstress test initialization interactive page 1500A includes a first user interaction element 1502, a first visual element 1504, a second visual element 1506, a third visual element 1508, and a second user interaction element 1512. Nonstress test initialization interactive page 1500A may, in some examples, be substantially the same as health check initialization interactive page 900C of FIG. 9C.

User interaction element 1502 may represent a back arrow that, when selected, will cause patient computing device 200 to display a previous interactive page on user interface element 222. In some examples, the previous interactive page may represent nonstress test information interactive page 1400 of FIG. 14. In some examples, the previous interactive page may represent test select interactive page 800A and/or test select interactive page 800B of FIGS. 8A-8B. In some examples, the previous interactive page may represent any interactive page that precedes nonstress test initialization interactive page 1500A in the nonstress test process of patient application 208. First visual element 1504 indicates that the system is initializing to prepare for a nonstress test. Second visual element 1506 includes a message instructing the patient to remain still without adjusting wearable device 150. Third visual element 1508 includes an avatar of the patient that displays a general location of wearable device 150 and sensors 152 on the avatar. Second user interaction element 1512 may redirect to a list of troubleshooting tips for achieving good contact between each sensor of sensors 152 and the patient.

FIG. 15B is a conceptual diagram illustrating a second nonstress test initialization interactive page 1500B, in accordance with one or more techniques of this disclosure. As seen in FIG. 15B, nonstress test initialization interactive page 1500B includes a first user interaction element 1502, a first visual element 1504, a second visual element 1506, a third visual element 1508, a fourth visual element 1510, and a second user interaction element 1512. First nonstress test initialization interactive page 1500A may, in some examples, be substantially the same as second nonstress test initialization interactive page 1500B, except that first visual element 1504 includes the text “WARM-UP PERIOD” instead of “HEALTH CHECK”, and second visual element 1506 of second nonstress test initialization interactive page 1500B includes a different message as compared with second visual element 1506 of first nonstress test initialization interactive page 1500A. Nonstress test initialization interactive page 1500B also includes fourth visual element 1510 that indicates an amount of time remaining in the warm-up period. In some examples, the warm-up period may last for three minutes, but this is not required. The warm-up period may last for any set amount of time. In any case, fourth visual element 1510 counts down to zero from the duration of the warm-up period.

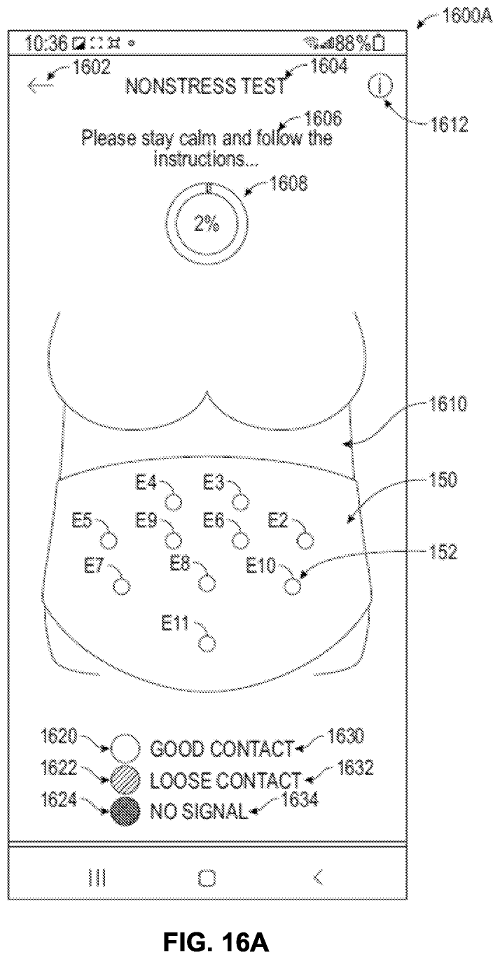

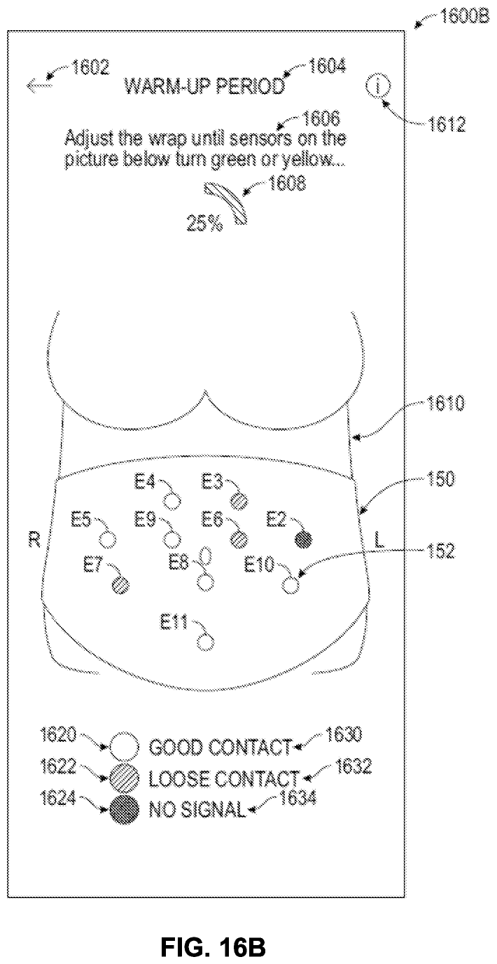

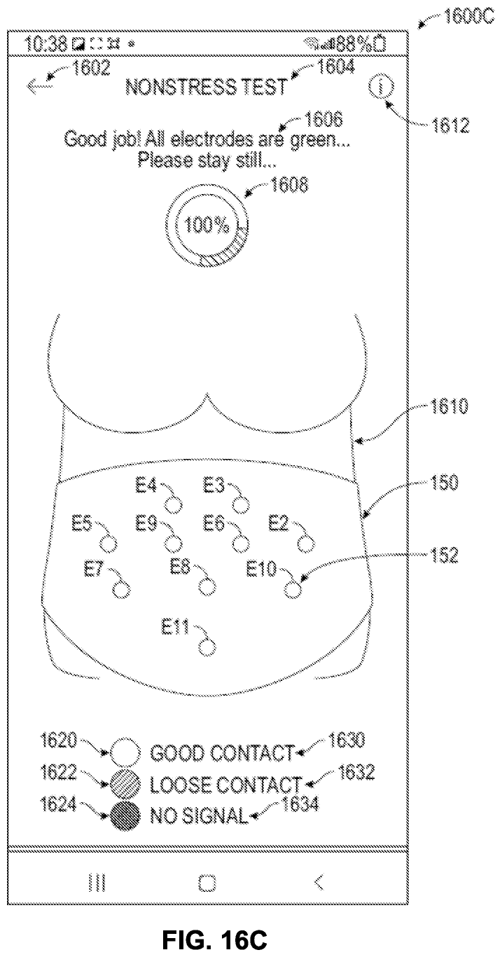

FIG. 16A is a conceptual diagram illustrating a first nonstress test sensor contact interactive page 1600A, in accordance with one or more techniques of this disclosure. As seen in FIG. 16A, first nonstress test sensor contact interactive page 1600A includes a first user interaction element 1602, a first visual element 1604, a second visual element 1606, a third visual element 1608, a fourth visual element 1610. First nonstress test sensor contact interactive page 1600A may include a second user interaction element 1612. FIG. 16A also includes a legend having a first indicator 1620, a second indicator 1622, a third indicator 1624, and a first descriptor 1630, a second descriptor 1632, and a third descriptor 1634.

User interaction element 1602 may represent a back arrow that, when selected, will cause patient computing device 200 to display a previous interactive page on user interface element 222. In some examples, the previous interactive page may represent nonstress test initialization interactive page 1500A of FIG. 15A. In some examples, the previous interactive page may represent nonstress test information interactive page 1400 of FIG. 14. In some examples, the previous interactive page may represent test select interactive page 800A and/or test select interactive page 800B of FIGS. 8A-8B. In some examples, the previous interactive page may represent any interactive page that precedes first nonstress test sensor contact interactive page 1600A in the nonstress test process of patient application 208.

First visual element 1604 indicates that first nonstress test sensor contact interactive page 1600A corresponds to a nonstress test. Second visual element 1606 includes a message instructing the patient to stay calm and follow instructions presented on user interface element 222 of computing device 200. Third visual element 1608 represents a progress wheel that indicates a progress of a sensor contact process.

As seen in FIG. 16A, first nonstress test sensor contact interactive page 1600A includes a fourth visual element 1610 that resembles an avatar of a patient. The patient avatar includes wearable device 150 and sensors 152. Each sensor of sensors 152 is shaded according to a quality of the contact between the respective sensor and the patient. For example, the legend of FIG. 16A shows that sensors shaded according to the color of first indicator 1620 have “GOOD CONTACT” with the patient as described by first descriptor 1630, sensors shaded according to the color of second indicator 1622 have “LOOSE CONTACT” with the patient as described by second descriptor 1632, and sensors shaded according to the color of third indicator 1624 have “NO SIGNAL” as described by third descriptor 1634. In some examples, “good contact” corresponds to a high quality of contact between the respective sensor and the patient, “loose contact” corresponds to a low quality of contact between the respective sensor and the patient, and “no signal” corresponds to no contact between the respective sensor ant the patient.

Sensors 152 may include sensors E2-E11 of FIG. 4. In some examples, sensors 152 may include one or more additional sensors that are not displayed on first nonstress test sensor contact interactive page 1600A. For example, sensors 152 may include sensors E1 and E12 of FIG. 4 located on a back side of wearable device 150. A front side of wearable device 150 is shown in FIG. 16A. In some examples, computing device 200 may be configured to display another separate interactive page that indicates a level of contact between sensors E1 and E12 and the patient. In some examples, computing device 200 may be configured to indicate the level of contact between sensors E1 and E12 and the patient on the same interactive page 1600A that indicates the level of contact between sensors E2-E11 and the patient. In some examples, computing device 200 may cause first nonstress test sensor contact interactive page 1600A to display the level of contact between sensors E1 and E12 using one or more visual elements to the side of the visual element 1610 resembling the avatar of the patient. As seen in FIG. 16A, interactive page 1600A indicates that sensors E2-E10 have good contact with the patient, and sensor E11 has loose contact with the patient.

In some examples, first nonstress test sensor contact interactive page 1600A includes a second user interaction element 1612 that redirects to a list of troubleshooting tips for achieving good contact between each sensor of sensors 152. For example, when computing device 200 receives a user selection of second user interaction element 1612 while first nonstress test sensor contact interactive page 1600A is displayed, computing device 200 may display another interactive page that provides one or more suggestions or “tips” for achieving good contact between each sensor of sensors 152. In some examples, when computing device 200 receives a user selection of second user interaction element 1612 while first nonstress test sensor contact interactive page 1600A is displayed, computing device 200 may display troubleshooting interactive page 1000B of FIG. 10B.

First nonstress test sensor contact interactive page 1600A may update the status of sensors 152 in real-time as the patient views user interface element 222 so that the patient may adjust wearable device 150 until the sensors have good contact with the patient. This feature may improve a quality of data from the nonstress test as compared with systems that do not inform the patient of sensor contact status. For example, if patient adjusts wearable device 150 to improve the contact of sensor E11 with the patient, computing device 200 may transition the icon corresponding to electrode E11 to indicate that electrode E11 has good contact with the patient (e.g., update the icon corresponding to electrode E11 to have the first indicator 1620 instead of the second indicator 1622).

FIG. 16B is a conceptual diagram illustrating a second nonstress test sensor contact interactive page 1600B, in accordance with one or more techniques of this disclosure. As seen in FIG. 16B, second health check sensor contact interactive page 1600B includes a first user interaction element 1602, a first visual element 1604, a second visual element 1606, a third visual element 1608, a fourth visual element 1610, and a second user interaction element 1612. FIG. 16B also includes a legend having a first indicator 1620, a second indicator 1622, a third indicator 1624, and a first descriptor 1630, a second descriptor 1632, and a third descriptor 1634.

In some examples, interactive page 1600B is substantially the same as interactive page 1600A except that first visual element 1604 includes the text “WARM-UP PERIOD” instead of the text “NONSTRESS TEST,” and third visual element 1608 indicates that the sensor contact process is 25% complete, and at least some of sensors E2-E11 are associated with different descriptors. For example, in FIG. 16B, senor E2 has “NO SIGNAL,” sensors E3, E6, and E7 have “LOOSE CONTACT” with the patient, and sensors E4, E5, E8, E9, E10, and E11 have “GOOD CONTACT” with the patient. In some examples, as any one or combination of sensors E2, E3, E6, and E7 improve their contact with the patient, the progress wheel of third visual element 1608 may increase from 25% to a higher percentage value. When sufficient contact between sensors 152 and the patient is achieved, the progress wheel of third visual element 1608 may reach 100% and the sensor contact process may be complete. In some examples, the icons on interactive page 1600B may update in real time based on the contact between the sensors and the patient improving and/or worsening.

FIG. 16C is a conceptual diagram illustrating a third nonstress test sensor contact interactive page 1600C, in accordance with one or more techniques of this disclosure. As seen in FIG. 16C, third nonstress test sensor contact interactive page 1600C includes a first user interaction element 1602, a first visual element 1604, a second visual element 1606, a third visual element 1608, a fourth visual element 1610. Third nonstress test sensor contact interactive page 1600C may include a second user interaction element 1612. FIG. 16C also includes a legend having a first indicator 1620, a second indicator 1622, a third indicator 1624, and a first descriptor 1630, a second descriptor 1632, and a third descriptor 1634. In some examples, third nonstress test sensor contact interactive page 1600C is substantially the same as first nonstress test sensor contact interactive page 1600A of FIG. 16A except that the progress wheel of third visual element 1608 on third nonstress test sensor contact interactive page 1600C indicates that full contact (e.g., 100% contact) has been achieved between sensors 152 and the patient, and each of electrodes E2-E11 have good contact with the patient.

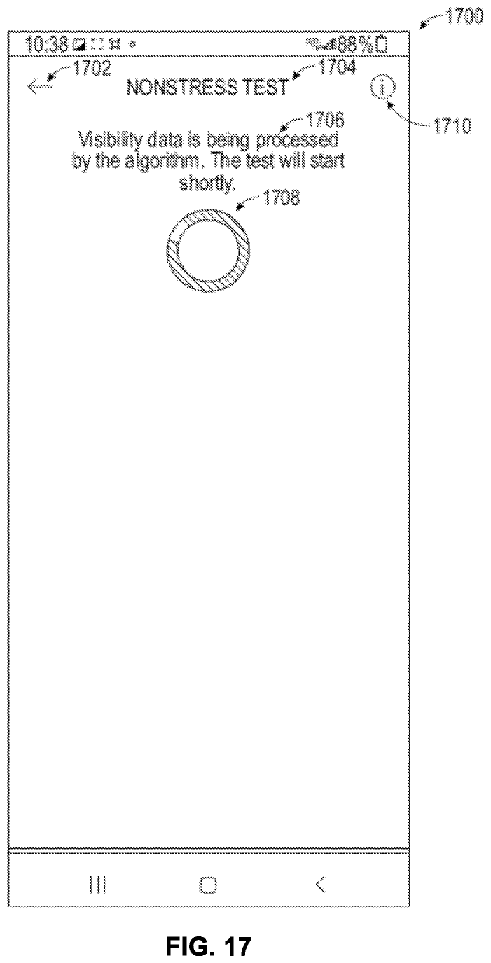

FIG. 17 is a conceptual diagram illustrating an example of a nonstress test visibility interactive page 1700, in accordance with one or more techniques of this disclosure. As seen in FIG. 17, nonstress test visibility interactive page 1700 includes a first user interaction element 1702, a first visual element 1704, a second visual element 1706, a third visual element 1708, and a second user interaction element 1710. First user interaction element 1702 may represent a back arrow that, when selected, will cause user interface element 222 of patient computing device 200 to display a previous interactive page. When computing device 200 receives a user selection of second user interaction element 1710 while nonstress test visibility interactive page 1700 is displayed, computing device 200 may display troubleshooting interactive page 1000B of FIG. 10B.

In some examples, nonstress test visibility interactive page 1700 may correspond to a yield test performed by computing device 200 and/or FMS 300. As described herein, a “yield test” may represent a test of signals collected by wearable device 150 via sensors 152. During a yield test, computing device 200 and/or FMS 300 may evaluate a quality, or “yield,” of one or more signals initially collected by wearable device 150 via sensors 152 after sensors 152 achieve good contact with the patient. If the yield test indicates that the quality of the one or more signals initially collected by wearable device 150 via sensors 152 does not meet a quality threshold, computing device 200 may display error interactive page 1000C of FIG. 10C. If the yield test indicates that the quality of the one or more signals initially collected by wearable device 150 via sensors 152 does meet a quality threshold, computing device 200 may proceed with a full nonstress test.

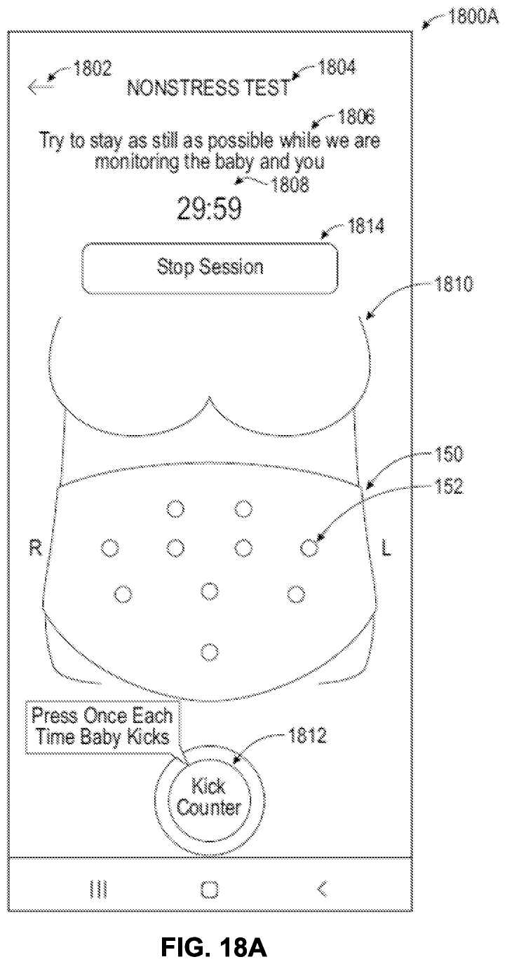

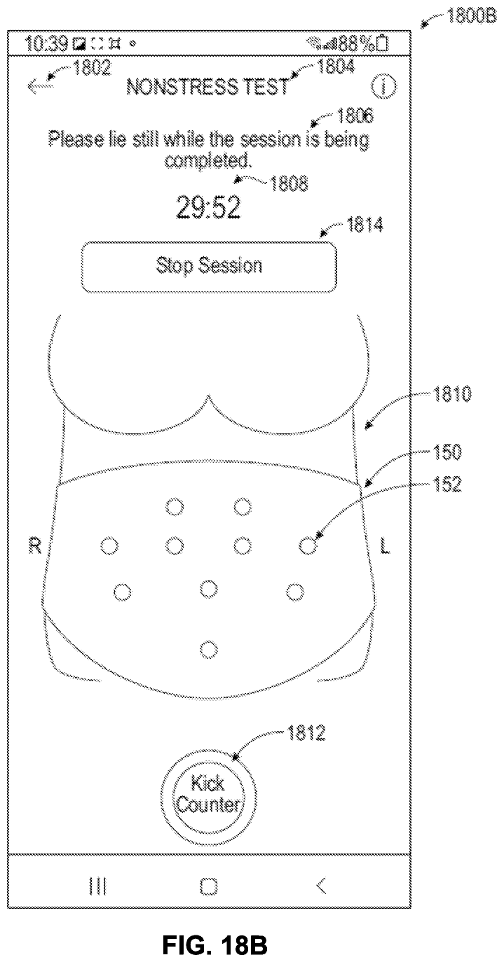

FIG. 18A is a conceptual diagram illustrating a first nonstress test progress interactive page 1800A, in accordance with one or more techniques of this disclosure. As seen in FIG. 18A, nonstress test progress interactive page 1800A includes a first user interaction element 1802, a first visual element 1804, a second visual element 1806, a third visual element 1808, a fourth visual element 1810, a second user interaction element 1812, and a third user interaction element 1814. In some examples, computing device 200 displays first nonstress test progress interactive page 1800A after completing a yield test while computing device 200 displays nonstress test visibility interactive page 1700 of FIG. 17. For example, computing device 200 may transition from displaying nonstress test visibility interactive page 1700 of FIG. 17 to displaying first nonstress test progress interactive page 1800A in response to determining that the yield test is sufficient. In some examples, computing device 200 displays first nonstress test progress interactive page 1800A in response to determining that sensors 152 have good contact with the patient. For example, computing device 200 may transition from displaying first nonstress test sensor contact interactive page 1600A, second nonstress test sensor contact interactive page 1600B and/or third nonstress test sensor contact interactive page 1600C to displaying first nonstress test progress interactive page 1800A in response to determining that at least a threshold number of sensors 152 have good contact with the patient.

First user interaction element 1802 may represent a back arrow that, when selected, will cause user interface element 222 of patient computing device 200 to display a previous interactive page. First visual element 1804 may include the words “NONSTRESS TEST” to indicate that a nonstress process is currently in progress. Second visual element 1806 may include a message instructing the patient to remain as still as possible while the nonstress test is in progress. Third visual element 1808 may include an amount of time that counts down to the end of the nonstress test. For example, a nonstress may last for an amount of time (e.g., 20 minutes, 30 minutes, 60 minutes, or any other amount of time). Third visual element 1108 may count down so that the patient is continuously informed as to how much longer the nonstress test will last. In the example of FIG. 18A, third visual element 1808 may display a time of 29:52, meaning that twenty-nine minutes and fifty-two seconds are remaining in the nonstress test.

Fourth visual element 1810 resembles an avatar of a patient. The patient avatar includes wearable device 150 and sensors 152. In some examples, each sensor of sensors 152 is shaded according to a quality of the contact between the respective sensor and the patient, but this is not required. In some examples, each sensor of sensors 152 represents a round icon that indicates a relative location of the respective sensor on the wearable device.

Second user interaction element 1812 may represent a “kick counter” for the user to press each time the baby kicks during the nonstress test. In some examples, it may be beneficial to periodically count the number of kicks that occur during a period of time (e.g., during a 30-minute nonstress test) in order to track a pregnancy. Tracking a rate of kicks in successive nonstress tests may indicate progress of a pregnancy. Second user interaction element 1812 may include an instruction for the patient to “press once each time baby kicks” in order to provide the patient with information as to how to conduct the nonstress test. Each time computing device 200 receives a user input to second user interaction element 1812, computing device 200 may increment a kick count. When the nonstress test is completed, computing device 200 may save the kick count associated with the nonstress test. Third user interaction element 1814 may represent a “stop session” button that, when pressed by the patient, causes computing device 200 to cease the nonstress test. For example, when computing device 200 receives an input to third user interaction element 1814, computing device 200 may cause wearable device 150 to stop collecting data for the nonstress test.

FIG. 18B is a conceptual diagram illustrating a nonstress test progress interactive page 1800B, in accordance with one or more techniques of this disclosure. As seen in FIG. 18B, nonstress test progress interactive page 1800B includes a first user interaction element 1802, a first visual element 1804, a second visual element 1806, a third visual element 1808, a fourth visual element 1810, a second user interaction element 1812, and a third user interaction element 1814. In some examples, the nonstress test progress interactive page 1800B is substantially the same as the nonstress test progress interactive page 1800A of FIG. 18A, except that nonstress test progress interactive page 1800B displays a different message on second visual element 1806 (e.g., “please lie still while the session is being completed” instead of “try to stay as still as possible while we are monitoring the baby and you”), and a different amount of time on third visual element 1108 (e.g., 29:52, instead of 29:59).

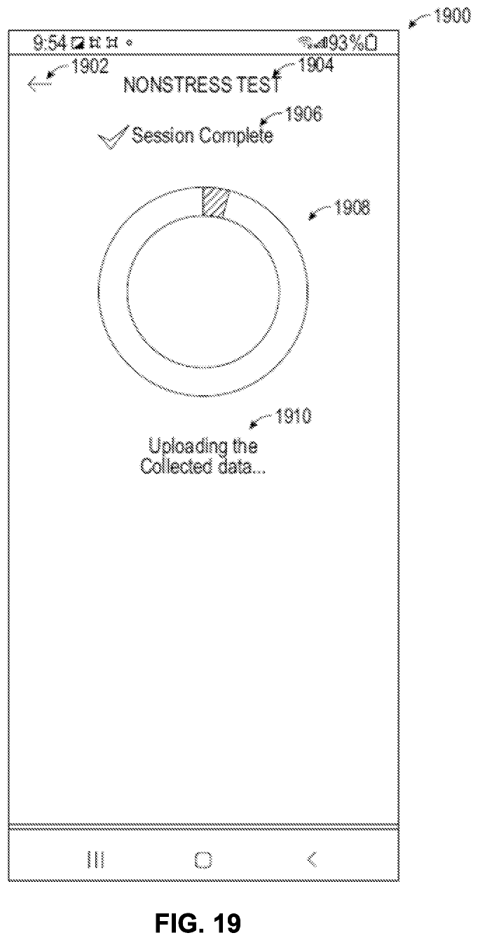

FIG. 19 is a conceptual diagram illustrating a nonstress test upload interactive page 1900, in accordance with one or more techniques of this disclosure. As seen in FIG. 19, nonstress test upload interactive page 1900 includes user interaction element 1902, a first visual element 1904, a second visual element 1906, a third visual element 1908, and a fourth visual element 1910.

First user interaction element 1902 may represent a back arrow that, when selected, will cause user interface element 222 of patient computing device 200 to display a previous interactive page. First visual element 1904 may include the words “NONSTRESS TEST” to indicate that computing device 200 is currently uploading nonstress test data. Second visual element 1906 may include the words “SESSION COMPLETE” to indicate that computing device 200 has finished collecting data for the nonstress test. Third visual element 1908 may include a progress wheel. In some examples, the progress wheel of third visual element 1908 may include a dark element and a light element. The dark element may expand around the progress wheel and the light element may shrink as the data is uploaded. Fourth visual element 1910 may include a message indicating that nonstress test data is being uploaded. In some examples, computing device 200 may upload the nonstress test data to FMS 300 for processing.

Computing device 200 may display nonstress test upload interactive page 1900A after the nonstress test is complete. For example, computing device 200 may display nonstress test upload interactive page 1900A when third visual element 1808 of FIGS. 18A-18B counts down to zero and the nonstress test is completed. That is, computing device 200 may transition from displaying interactive page 1800A and/or interactive page 1800B to displaying interactive page 1900 when the nonstress test is complete and data is ready for upload to FMS 300.

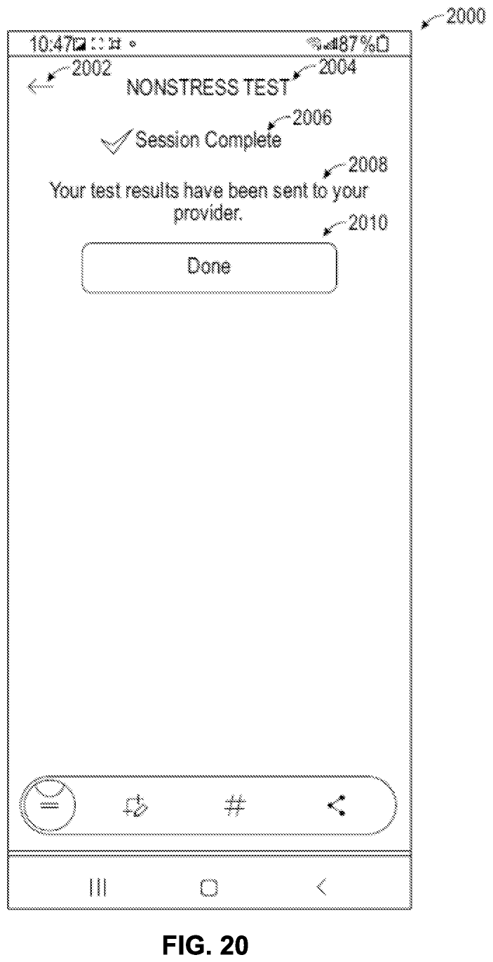

FIG. 20 is a conceptual diagram illustrating a nonstress test complete interactive page 2000, in accordance with one or more techniques of this disclosure. As seen in FIG. 20, nonstress test complete interactive page 2000 includes user interaction element 2002, a first visual element 2004, a second visual element 2006, a third visual element 2008, and a user interaction element 2010.

First user interaction element 2002 may represent a back arrow that, when selected, will cause user interface element 222 of patient computing device 200 to display a previous interactive page. First visual element 2004 may include the words “NONSTRESS TEST” to indicate that the interactive page corresponds to a nonstress test. Second visual element 2006 may include the words “SESSION COMPLETE” to indicate that computing device 200 has finished collecting data for the nonstress test. Third visual element 1908 may include a message indicating that the results of the nonstress test have been uploaded to the patient's provider (e.g., to FMS 300). Second user interaction element 2010 may represent a button that, when pressed, causes the computing device 200 to end the nonstress test. In some examples, computing device 200 may perform a yield check of the data collected during the nonstress test.

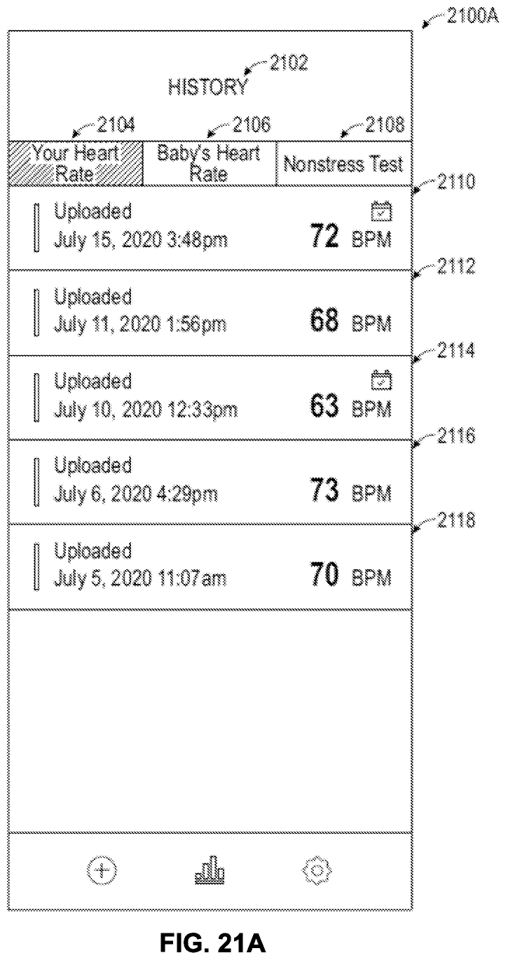

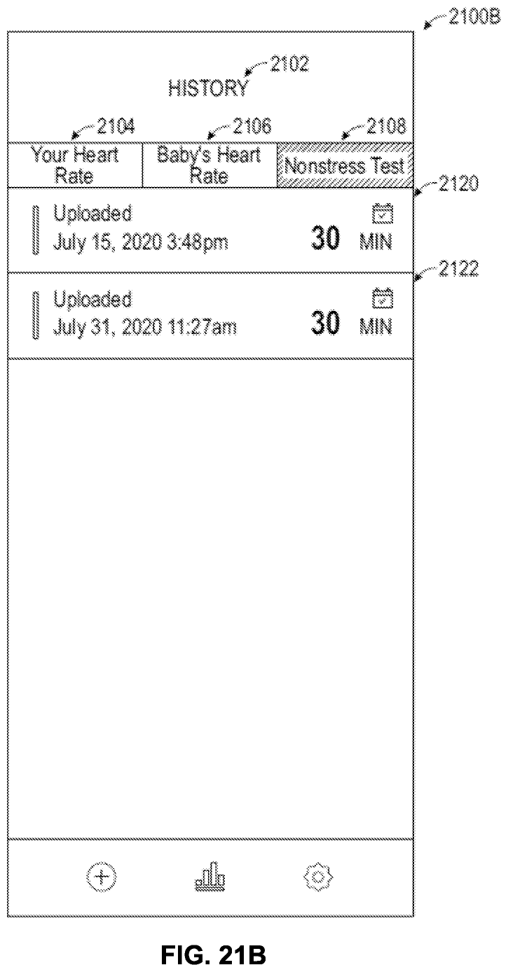

FIG. 21A is a conceptual diagram illustrating a maternal heart rate patient data history interactive page 2100A, in accordance with one or more techniques of this disclosure. Patient data history interactive page 2100A includes a visual element 2102, a first tab 2104, a second tab 2106, and a third tab 2108. In some examples, the first tab 2104 may be selected in the example of FIG. 21A. Visual element 2102 may indicate that interactive page 2100A includes patient data history. First tab 2104 may include a set of maternal heart rate entries 2110-2118, wherein each maternal heart rate entry of the set of maternal heart rate entries corresponds to a respective health check of a set of health checks. Second tab 2106 may, in some examples, include a list of fetal heart rate entries. Third tab 2108 may, in some examples, include a list of nonstress test entries. In some examples, computing device 200 may display maternal heart rate patient data history interactive page 2100A in response to receiving a user request to view patient data within the patient application 208.

Since first tab 2104 is selected in the example of FIG. 21A, maternal heart rate patient data history interactive page 2100A displays entries 2110-2118, each indicating a maternal heart rate and a health check corresponding to the maternal heart rate. For example, first maternal heart entry 2110 indicates a maternal heart rate of 72 BPM corresponding to a Jul. 15, 2020 health check, second entry 2112 indicates a maternal heart rate of 68 BPM corresponding to a Jul. 11, 2020 health check, third entry 2114 indicates a maternal heart rate of 63 BPM corresponding to a Jul. 10, 2020 health check, fourth entry 2116 indicates a maternal heart rate of 73 BPM corresponding to a Jul. 6, 2020 health check, and third entry 2118 indicates a maternal heart rate of 70 BPM corresponding to a Jul. 5, 2020 health check. In some examples, computing device 200 may transition to displaying a set of fetal heart rate entries in response to receiving a user input to second tab 2106. In some examples, computing device 200 may transition to displaying a set of nonstress test entries in response to receiving a user input to third tab 2108.

FIG. 21B is a conceptual diagram illustrating a nonstress test history interactive page 2100B, in accordance with one or more techniques of this disclosure. Nonstress test history interactive page 2100B includes a visual element 2102, a first tab 2104, a second tab 2106, and a third tab 2108. In some examples, the third tab 2108 may be selected in the example of FIG. 21A. Visual element 2102 may indicate that interactive page 2100A includes patient data history. Third tab 2108 may include a set of nonstress entries 2120-2122, wherein each nonstress test entry of the set of nonstress test entries corresponds to a respective nonstress tests of a set of nonstress tests. First tab 2104 may, in some examples, include a list of maternal heart rate entries. Second tab 2106 may, in some examples, include a list of fetal heart rate entries. In some examples, computing device 200 may display maternal heart rate patient data history interactive page 2100A in response to receiving a user request to view patient data within the patient application 208.

Since third tab 2108 is selected in the example of FIG. 21B, nonstress test history interactive page 2100B displays entries 2120-2122, each corresponding to a nonstress test of a set of nonstress tests. For example, nonstress test entry 2120 indicates that a 30-minute nonstress test was completed on Jul. 15,2020, and nonstress test entry 2122 indicates that a 30-minute nonstress test was completed on Jul. 31,2020. In some examples, computing device 200 may transition to displaying a set of maternal heart rate entries in response to receiving a user input to first tab 2104. In some examples, computing device 200 may transition to displaying a set of fetal heart rate entries in response to receiving a user input to second tab 2106.

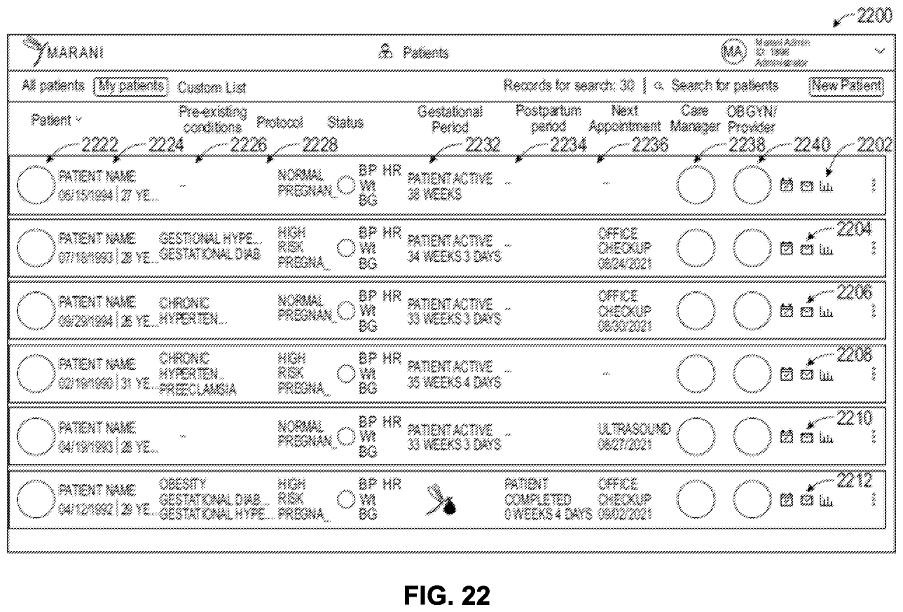

FIGS. 22-27 are conceptual diagrams illustrating example interactive pages that may be provided for display on one or more user interfaces of a provider computing system (such as provider computing system 180) by provider portal 182, in accordance with one or more techniques of this disclosure.

FIG. 22 is a conceptual diagram illustrating an interactive page 2200 including a list of patients for display by provider portal 182, in accordance with one or more techniques of this disclosure. In some examples, a clinician may interact with provider portal 182 to view information and data corresponding to one or more patients. The interactive page 2200 may, in some examples, include a list of entries 2202-2212. Each entry of the list of entries 2202-2212 may correspond to a patient. For example, first entry 2202 may correspond to a first patient, second entry 2204 may correspond to a second patient, third entry 2206 may correspond to a third patient, fourth entry 2208 may correspond to a fourth patient, fifth entry 2210 may correspond to a fifth patient, and sixth entry 2212 may correspond to a sixth patient.

Each entry of entries 2202-2212 may include information in one or more of categories 2222-2240. Categories 2222-2240 include a photograph category 2222, a name and birthdate category 2224, a pre-existing conditions category 2226, a protocol category 2228, a patient status category 2230, a gestational period category 2232, a postpartum period category 2234, a next appointment category 2236, a care manager category 2238, and a clinician category 2240. The photograph category 2222 may include a photograph of the patient corresponding to the respective entry. The patient name and birthdate category 2224 may include the name and birthdate of the patient corresponding to the respective entry.

In some examples, the pre-existing conditions category 2226 may include one or more pre-existing conditions corresponding to the patient for the respective entry. If a patient does not have pre-existing conditions, then the pre-existing conditions category 2226 may be blank for that patient. For example, the patient for the first entry 2202 does not have any pre-existing conditions listed under the pre-existing conditions category 2226, but the patent for the fourth entry 2208 has chronic hypertension and preeclampsia listed under the pre-existing conditions category 2226. The protocol category 2228 may indicate a level of risk corresponding to each patient. For example, the protocol category 2228 indicates that the patient corresponding to entry 2202 has a “normal pregnancy,” whereas the protocol category 2228 indicates that the patient corresponding to entry 2212 has a “high-risk pregnancy.” In some examples, a clinician may set a risk level for each patient based on the clinician's assessment of each patient's medical history and needs. In some examples, there may be risk levels other than “normal” and “high-risk.”

The gestational period category 2232 may include information indicating a patient's progress through a pregnancy. For example, the gestational period category 2232 indicates that the patient corresponding to entry 2202 is 38 weeks into pregnancy, and the gestational period category 2232 indicates that the patient corresponding to entry 2202 is 33 weeks and 3 days into pregnancy. The patient corresponding to entry 2212 has given birth, so the gestational period category 2232 is empty. The postpartum period category 2234 may include information indicating a patient's progress after pregnancy. The patients corresponding to entries 2202-2210 are still pregnant, so the postpartum period category 2234 is empty for these patients. The patient corresponding to entry 2212 has given birth, and the postpartum period category 2234 indicates that the patient completed pregnancy four days ago. The next appointment category 2236 may indicate a date of a patient's next scheduled appointment. The care manager category 2238 may include a photograph of a care manager working with each patient. The clinician category 2240 may include a photograph of a clinician working with each patient

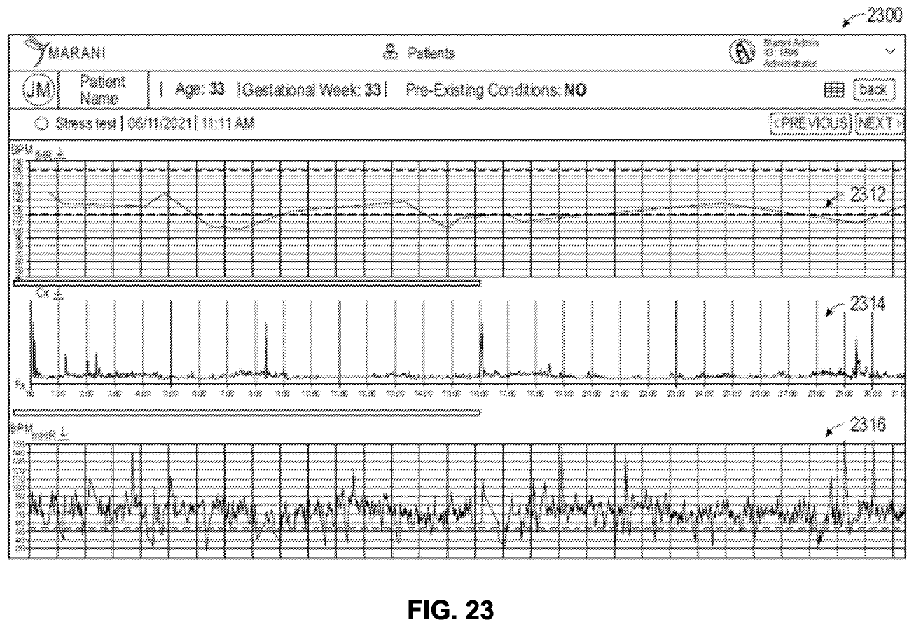

FIG. 23 is a conceptual diagram illustrating an interactive page 2300 including data corresponding to a patient monitoring session, in accordance with one or more techniques of this disclosure. In some examples, a clinician may navigate to interactive page 2300 by selecting one of the patients listed on interactive page 2200 of FIG. 22. As seen in FIG. 23, interactive page 2300 includes information corresponding to a patient including a name, an age, a gestational week, and a YES/NO indication of pre-existing conditions. Interactive page 2300 includes a first plot 2312, a second plot 2314, and a third plot 2316. In some examples, the first plot 2312 represents a fetal heart rate over the course of a health check, the second plot 2314 represents a combined electrocardiogram (ECG) over the course of the health check, and the third plot 2316 represents a maternal heart rate over the course of the health check.

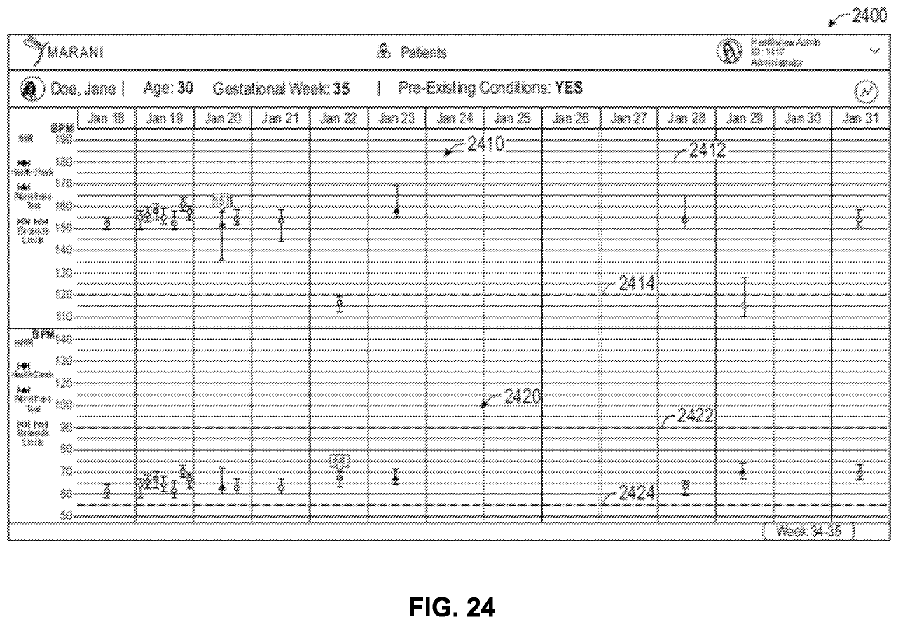

FIG. 24 is a conceptual diagram illustrating an interactive page 2400 including data corresponding to a set of nonstress tests, in accordance with one or more techniques of this disclosure. In some examples, a clinician may navigate to interactive page 2400 by selecting one of the patients listed on interactive page 2200 of FIG. 22. As seen in FIG. 24, interactive page 2400 includes information corresponding to a patient including a name, an age, a gestational week, and a YES/NO indication of pre-existing conditions. In some examples, each nonstress test of the set of nonstress tests includes a range of fetal heart rates, a mean fetal heart rate, a range of maternal heart rates, and a mean maternal heart rate. Plot 2420 indicates the range of fetal heart rates and the mean fetal heart rate corresponding to each nonstress test of the set of nonstress tests. Plot 2420 indicates the range of maternal heart rates and the mean maternal heart rate corresponding to each nonstress test of the set of nonstress tests. Plot 2410 includes an upper-bound fetal heart rate 2412 and a lower-bound fetal heart rate 2414. Plot 2420 includes an upper-bound maternal heart rate 2422 and a lower-bound maternal heart rate 2424. The upper-bound and lower-bound heart rates may indicate expected ranges of heart rates.

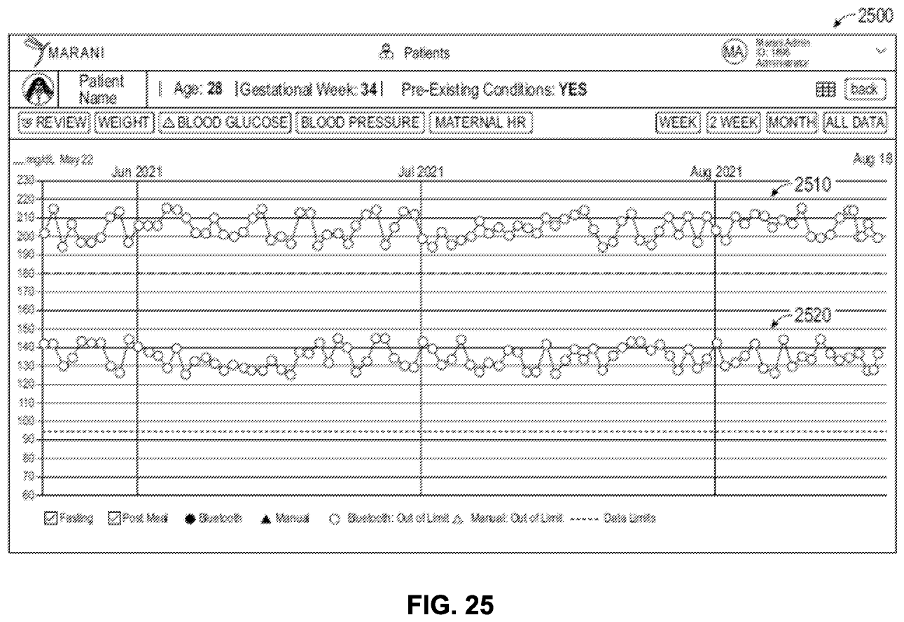

FIG. 25 is a conceptual diagram illustrating an interactive page 2500 including data corresponding to a set of patient glucose level tests, in accordance with one or more techniques of this disclosure. In some examples, a clinician may navigate to interactive page 2500 by selecting one of the patients listed on interactive page 2200 of FIG. 22. As seen in FIG. 25, interactive page 2500 includes information corresponding to a patient including a name, an age, a gestational week, and a YES/NO indication of pre-existing conditions. In some examples, plot 2510 includes a set of post-meal glucose level tests. In some examples, plot 2520 includes a set of fasting glucose level tests.

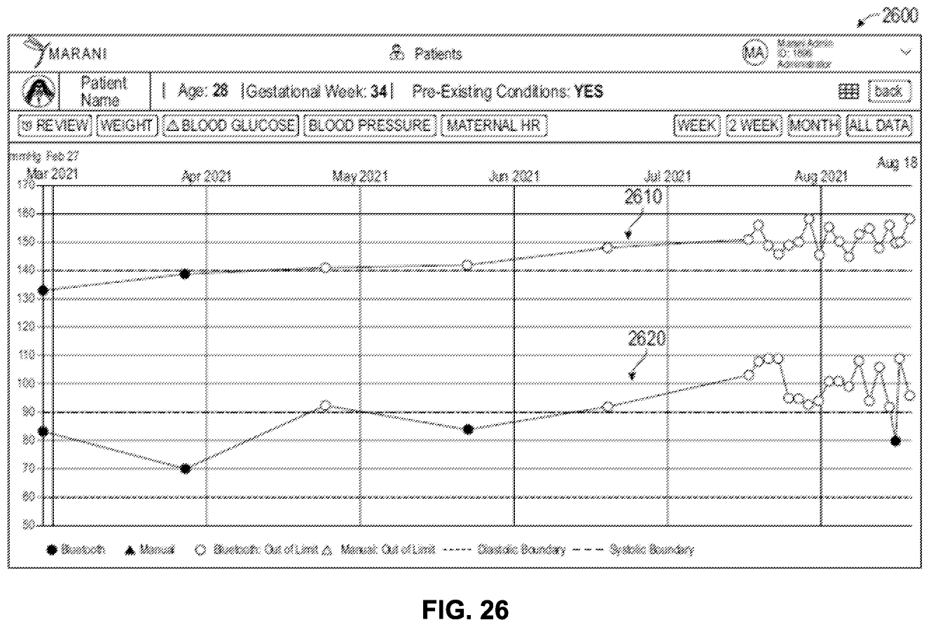

FIG. 26 is a conceptual diagram illustrating an interactive page 2600 including data corresponding to a set of patient blood pressure tests, in accordance with one or more techniques of this disclosure. In some examples, a clinician may navigate to interactive page 2600 by selecting one of the patients listed on interactive page 2200 of FIG. 22. As seen in FIG. 26, interactive page 2600 includes information corresponding to a patient including a name, an age, a gestational week, and a YES/NO indication of pre-existing conditions. Plot 2610 may include systolic blood pressure values, and plot 2620 may include diastolic blood pressure values.

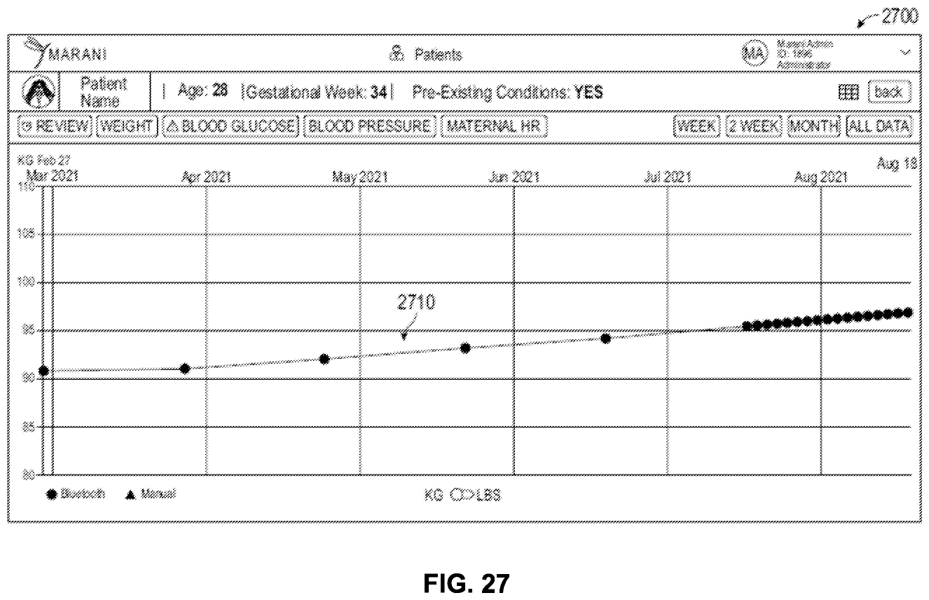

FIG. 27 is a conceptual diagram illustrating an interactive page 2700 including data corresponding to a patient's weight, in accordance with one or more techniques of this disclosure. Plot 2710 of interactive page 270 indicates a patient's weight over a period of time (e.g., a period of six months). In some examples, a clinician may navigate to interactive page 2700 by selecting one of the patients listed on interactive page 2200 of FIG. 22. As seen in FIG. 27, interactive page 2700 includes information corresponding to a patient including a name, an age, a gestational week, and a YES/NO indication of pre-existing conditions.

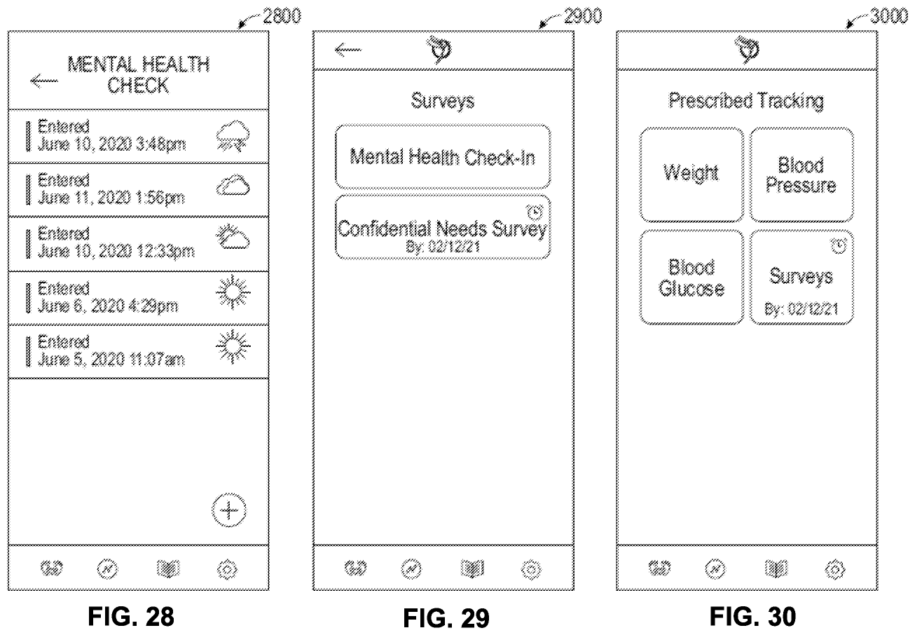

FIGS. 28-30 are conceptual diagrams illustrating interactive pages for display by patient computing device 200, in accordance with one or more techniques of this disclosure. Each interactive page of the interactive pages shown in FIGS. 28-30 may include one or more user interface elements and one or more visual elements. A user may be able to provide one or more user inputs to the interface elements, and the one or more visual elements may provide information to the user. FIG. 28 shows an interactive page 2800 displaying entries corresponding to one or more mental health assessments conducted by the patient. FIG. 29 shows a “Surveys” interactive page 2900 displaying one or more user interface elements by which a user may initiate a “Mental Health Check-In” session or a “Confidential Needs Survey” session, respectively. FIG. 30 shows a “Prescribed Tracking” interactive page 3000 displaying one or more user interface elements by which a user may initiate data entry or monitoring of weight, blood pressure, blood glucose, or access one or more surveys, respectively. The “Surveys” user interface element displays a date by which the patient is to complete the required survey accessible via the “Surveys” user interface element.

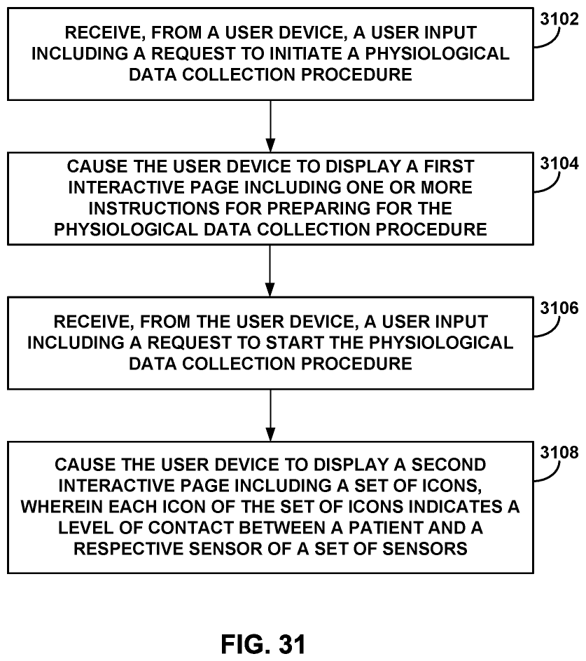

FIG. 31 is a flow diagram illustrating an example operation for using an application to ensure a strong connection between sensors of a medical device and a patient, in accordance with one or more techniques of this disclosure. FIG. 31 is described with respect to wearable device 150, sensors 152, and computing device 200 of FIGS. 1-2. However, the techniques of FIG. 31 may be performed by different components of wearable device 150, sensors 152, and computing device 200 or by additional or alternative medical device systems.

Computing device 200 may receive a user input including a request to initiate a physiological data collection procedure (3102). In some examples, the user input may include a request to initiate a health check. In some examples, the user input may include a request to initiate a nonstress test. In some examples, a health check may represent an analysis of a brief segment (e.g., 5 minutes) of physiological data collected by wearable device 150. The analysis of the health check may include determining fetal heart rate, maternal heart rate, or other physiological parameters. In some examples, a nonstress test may represent an analysis of a longer segment (e.g., 30 minutes) of physiological data collected by wearable device 150. In some examples, the nonstress test may include analyzing physiological data collected via sensors 152 of wearable device 150 and/or a kick count. The kick count may be based on user input indicating each time that the baby kicks during the nonstress test.

Computing device 200 may display a first interactive page including one or more instructions for preparing for the physiological data collection procedure (3104). In some examples, the one or more instructions may include instructions for the patient to remain still during the physiological data collection procedure. In some examples, the one or more instructions may include instructions for the patient to adjust the wearable device 150 to achieve a stronger level of contact between sensors 152 and the patient. Computing device 200 may receive a user input including a request to start the physiological data collection procedure (3106). Computing device 200 may display a second interactive page including a set of icons, wherein each icon of the set of icons indicates a level of contact between a patient and a respective sensor of a set of sensors 152 (3108).

The following numbered clauses may demonstrate one or more aspects of the disclosure.

Clause 1: A system comprising: a memory; and one or more processors in communication with the memory, wherein the one or more processors are configured to: receive, from a user device, a user input including a request to initiate a physiological data collection procedure; cause the user device to display a first interactive page including one or more instructions for preparing for the physiological data collection procedure; receive, from the user device, a user input including a request to start the physiological data collection procedure; and cause the user device to display a second interactive page including a set of icons, wherein each icon of the set of icons corresponds to a sensor of a set of sensors on a wearable device of the patient, and wherein each icon of the set of icons indicates a level of contact between a patient and the respective sensor of the set of sensors.

Clause 2: The system of clause 1, wherein the level of contact may include, for each sensor of the set of sensors, good contact, loose contact, or no contact, and wherein the one or more processors are configured to: for each sensor of the set of sensors, determine one or more metrics indicative of a signal quality of a biopotential signal acquired by the sensor; for each sensor of the set of sensors, determine, based on the one or more metrics indicative of the signal quality of the biopotential signal acquired by the sensor, whether the sensor has good contact with the patient, loose contact with the patient, or no contact with the patient; and cause the second interactive page displayed by the user device to indicate the level of contact corresponding to each sensor of the set of sensors.

Clause 3: The system of clause 2, wherein the one or more metrics include one or more of a signal-to-noise ratio, a noise-to-noise ratio, or a signal strength of the biopotential signal acquired by the sensor.

Clause 4: The system of clause 2, wherein the one or more processors are configured to: identify a number of sensors of the set of sensors that have no contact with the patient; identify a number of sensors of the set of sensors that have loose contact with the patient; and determine whether a sum of the number of sensors that have no contact and the number of sensors that have loose contact is greater than a threshold number of sensors.

Clause 5: The system of clause 4, wherein based on determining that the number of sensors is greater than the threshold number of sensors, the one or more processors are configured to: cause the second interactive page displayed by the user device to identify each sensor of the set of sensors that has no contact with the patient; cause the second interactive page displayed by the user device to identify each sensor of the set of sensors that has loose contact with the patient; and cause the second interactive page displayed by the user device to identify each sensor of the set of sensors that has good contact with the patient.

Clause 6: The system of clause 4, wherein based on determining that the number of sensors is not greater than the threshold number of sensors, the one or more processors are configured to: cause the user device to display a third interactive page corresponding to a performance of the physiological data collection procedure, wherein the third interactive page indicates an amount of time remaining in the physiological data collection procedure; and control the wearable device to perform the physiological data collection procedure by collecting one or more physiological signals via the set of sensors.

Clause 7: The system of clause 6, wherein when the physiological data collection procedure is complete, the one or more processors are configured to: cause the user device to display a fourth interactive page which indicates that results from the physiological data collection procedure is uploading for analysis; and upload the results from the physiological data collection procedure to a fetal monitoring system for analysis.

Clause 8: The system of clause 1, wherein the one or more processors are further configured to: receive a user input selecting the physiological data collection procedure from a set of physiological data collection procedures, wherein the set of physiological data collection procedures include a health check procedure and a nonstress test procedure; and cause the user device to display the first interactive page based on receiving the user input.

Clause 9: The system of clause 8, wherein the one or more processors are further configured to: receive the user input selecting the health check procedure; and control, based on the level of contact between the patient and each sensor of the set of sensors, the wearable device to perform the health check procedure by collecting one or more physiological signals from the patient, wherein the one or more physiological signals indicate a maternal cardiac activity and a fetal cardiac activity.

Clause 10: The system of clause 8, wherein the one or more processors are further configured to: receive the user input selecting the nonstress test; and control, based on the level of contact between the patient and each sensor of the set of sensors, the wearable device to perform the nonstress by collecting one or more physiological signals from the patient, wherein the one or more physiological signals indicate a health of the patient's pregnancy.

Clause 11: A method comprising: receiving, by one or more processors from a user device, a user input including a request to initiate a physiological data collection procedure, wherein the one or more processors are in communication with the memory; causing, by the one or more processors, the user device to display a first interactive page including one or more instructions for preparing for the physiological data collection procedure; receiving, by the one or more processors from the user device, a user input including a request to start the physiological data collection procedure; and causing, by the one or more processors, the user device to display a second interactive page including a set of icons, wherein each icon of the set of icons corresponds to a sensor of a set of sensors on a wearable device of the patient, and wherein each icon of the set of icons indicates a level of contact between a patient and the respective sensor of the set of sensors.

Clause 12: The method of clause 11, wherein the level of contact may include, for each sensor of the set of sensors, good contact, loose contact, or no contact, and wherein the method further comprises: for each sensor of the set of sensors, determining one or more metrics indicative of a signal quality of a biopotential signal acquired by the sensor; for each sensor of the set of sensors, determining, based on the one or more metrics indicative of the signal quality of the biopotential signal acquired by the sensor, whether the sensor has good contact with the patient, loose contact with the patient, or no contact with the patient; and causing the second interactive page displayed by the user device to indicate the level of contact corresponding to each sensor of the set of sensors.

Clause 13: The method of clause 12, wherein the one or more metrics include one or more of a signal-to-noise ratio, a noise-to-noise ratio, or a signal strength of the biopotential signal acquired by the sensor.

Clause 14: The method of claim 12, wherein the method further comprises: identifying, by the one or more processors, a number of sensors of the set of sensors that have no contact with the patient; identifying, by the one or more processors, a number of sensors of the set of sensors that have loose contact with the patient; and determining, by the one or more processors, whether a sum of the number of sensors that have no contact and the number of sensors that have loose contact is greater than a threshold number of sensors.

Clause 15: The method of clause 14, wherein based on determining that the number of sensors is greater than the threshold number of sensors, method further comprises: causing, by the one or more processors, the second interactive page displayed by the user device to identify each sensor of the set of sensors that has no contact with the patient; causing, by the one or more processors, the second interactive page displayed by the user device to identify each sensor of the set of sensors that has loose contact with the patient; and causing, by the one or more processors, the second interactive page displayed by the user device to identify each sensor of the set of sensors that has good contact with the patient.

Clause 16: The method of clause 14, wherein based on determining that the number of sensors is not greater than the threshold number of sensors, the method further comprises: causing, by the one or more processors, the user device to display a third interactive page corresponding to a performance of the physiological data collection procedure, wherein the third interactive page indicates an amount of time remaining in the physiological data collection procedure; and controlling, by the one or more processors, the wearable device to perform the physiological data collection procedure by collecting one or more physiological signals via the set of sensors.

Clause 17: The method of clause 16, wherein when the physiological data collection procedure is complete, the method further comprises: causing, by the one or more processors, the user device to display a fourth interactive page which indicates that results from the physiological data collection procedure is uploading for analysis; and uploading, by the one or more processors, the results from the physiological data collection procedure to a fetal monitoring system for analysis.

Clause 18: The method of clause 11, further comprising: receiving, by the one or more processors, a user input selecting the physiological data collection procedure from a set of physiological data collection procedures, wherein the set of physiological data collection procedures include a health check procedure and a nonstress test procedure; and causing, by the one or more processors, the user device to display the first interactive page based on receiving the user input.

Clause 19: The method of clause 18, further comprising: receiving, by the one or more processors, the user input selecting the health check procedure; and controlling, by the one or more processors based on the level of contact between the patient and each sensor of the set of sensors, the wearable device to perform the health check procedure by collecting one or more physiological signals from the patient, wherein the one or more physiological signals indicate a maternal cardiac activity and a fetal cardiac activity.

Clause 20: A non-transitory computer-readable medium comprising instructions for causing one or more processors to: receive, from a user device, a user input including a request to initiate a physiological data collection procedure; cause the user device to display a first interactive page including one or more instructions for preparing for the physiological data collection procedure; receive, from the user device, a user input including a request to start the physiological data collection procedure; and cause the user device to display a second interactive page including a set of icons, wherein each icon of the set of icons corresponds to a sensor of a set of sensors on a wearable device of the patient, and wherein each icon of the set of icons indicates a level of contact between a patient and the respective sensor of the set of sensors.

Additional examples of components, devices, apparatus, methods, and/or systems which may be used in connection with one or more aspects of this disclosure are described in U.S. Pat. No. 9,579,055, issued Feb. 28, 2017, U.S. Pat. No. 10,292,652, issued May 21, 2019, and United States Patent Application Publication No. 2020/0113470, published on Apr. 16, 2020, both of which are incorporated herein by reference in their entirety.

In one or more examples, the functions described may be implemented in any combination of processing circuitry, including hardware, software, firmware, or any combination thereof. If implemented in software, the functions may be stored on or transmitted over a computer-readable medium as one or more instructions or code and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can include RAM, ROM, EEPROM, CD-ROM or other optical disk storage, magnetic disk storage, or other magnetic storage devices, flash memory, or any other medium that can be used to store program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more DSPs, general purpose microprocessors and/or microcontrollers, ASICs, FPGAs, or other equivalent integrated or discrete logic circuitry, as well as any combination of such components. Accordingly, the term “processor,” as used herein may refer to any of the foregoing structures or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless communication device, a microprocessor, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware, and/or any other type or combination of processing circuitry.

Various examples have been described. These and other examples are within the scope of the following claims.

What is claimed is:

1. A system comprising:
a memory; and
one or more processors in communication with the memory, wherein the one or more processors are configured to:
receive, from a user device, a user input including a request to initiate a physiological data collection procedure;
cause the user device to display a first interactive page including one or more instructions for preparing for the physiological data collection procedure;
receive, from the user device, a user input including a request to start the physiological data collection procedure; and
cause the user device to display a second interactive page including a set of icons, wherein each icon of the set of icons corresponds to a sensor of a set of sensors on a wearable device of the patient, and wherein each icon of the set of icons indicates a level of contact between a patient and the respective sensor of the set of sensors.

2. The system of claim 1, wherein the level of contact may include, for each sensor of the set of sensors, good contact, loose contact, or no contact, and wherein the one or more processors are configured to:
for each sensor of the set of sensors, determine one or more metrics indicative of a signal quality of a biopotential signal acquired by the sensor;
for each sensor of the set of sensors, determine, based on the one or more metrics indicative of the signal quality of the biopotential signal acquired by the sensor, whether the sensor has good contact with the patient, loose contact with the patient, or no contact with the patient; and
cause the second interactive page displayed by the user device to indicate the level of contact corresponding to each sensor of the set of sensors.

3. The system of claim 2, wherein the one or more metrics include one or more of a signal-to-noise ratio, a noise-to-noise ratio, or a signal strength of the biopotential signal acquired by the sensor.

4. The system of claim 2, wherein the one or more processors are configured to:
identify a number of sensors of the set of sensors that have no contact with the patient;
identify a number of sensors of the set of sensors that have loose contact with the patient; and
determine whether a sum of the number of sensors that have no contact and the number of sensors that have loose contact is greater than a threshold number of sensors.

5. The system of claim 4, wherein based on determining that the number of sensors is greater than the threshold number of sensors, the one or more processors are configured to:
cause the second interactive page displayed by the user device to identify each sensor of the set of sensors that has no contact with the patient;
cause the second interactive page displayed by the user device to identify each sensor of the set of sensors that has loose contact with the patient; and
cause the second interactive page displayed by the user device to identify each sensor of the set of sensors that has good contact with the patient.

6. The system of claim 4, wherein based on determining that the number of sensors is not greater than the threshold number of sensors, the one or more processors are configured to:
cause the user device to display a third interactive page corresponding to a performance of the physiological data collection procedure, wherein the third interactive page indicates an amount of time remaining in the physiological data collection procedure; and
control the wearable device to perform the physiological data collection procedure by collecting one or more physiological signals via the set of sensors.

7. The system of claim 6, wherein when the physiological data collection procedure is complete, the one or more processors are configured to:
cause the user device to display a fourth interactive page which indicates that results from the physiological data collection procedure is uploading for analysis; and
upload the results from the physiological data collection procedure to a fetal monitoring system for analysis.

8. The system of claim 1, wherein the one or more processors are further configured to:
receive a user input selecting the physiological data collection procedure from a set of physiological data collection procedures, wherein the set of physiological data collection procedures include a health check procedure and a nonstress test procedure; and
cause the user device to display the first interactive page based on receiving the user input.

9. The system of claim 8, wherein the one or more processors are further configured to:
receive the user input selecting the health check procedure; and
control, based on the level of contact between the patient and each sensor of the set of sensors, the wearable device to perform the health check procedure by collecting one or more physiological signals from the patient, wherein the one or more physiological signals indicate a maternal cardiac activity and a fetal cardiac activity.

10. The system of claim 8, wherein the one or more processors are further configured to:
receive the user input selecting the nonstress test; and
control, based on the level of contact between the patient and each sensor of the set of sensors, the wearable device to perform the nonstress by collecting one or more physiological signals from the patient, wherein the one or more physiological signals indicate a health of the patient's pregnancy.

11. A method comprising:
receiving, by one or more processors from a user device, a user input including a request to initiate a physiological data collection procedure, wherein the one or more processors are in communication with the memory;
causing, by the one or more processors, the user device to display a first interactive page including one or more instructions for preparing for the physiological data collection procedure;
receiving, by the one or more processors from the user device, a user input including a request to start the physiological data collection procedure; and
causing, by the one or more processors, the user device to display a second interactive page including a set of icons, wherein each icon of the set of icons corresponds to a sensor of a set of sensors on a wearable device of the patient, and wherein each icon of the set of icons indicates a level of contact between a patient and the respective sensor of the set of sensors.

12. The method of claim 11, wherein the level of contact may include, for each sensor of the set of sensors, good contact, loose contact, or no contact, and wherein the method further comprises:
for each sensor of the set of sensors, determining one or more metrics indicative of a signal quality of a biopotential signal acquired by the sensor;
for each sensor of the set of sensors, determining, based on the one or more metrics indicative of the signal quality of the biopotential signal acquired by the sensor, whether the sensor has good contact with the patient, loose contact with the patient, or no contact with the patient; and
causing the second interactive page displayed by the user device to indicate the level of contact corresponding to each sensor of the set of sensors.

13. The method of claim 12, wherein the one or more metrics include one or more of a signal-to-noise ratio, a noise-to-noise ratio, or a signal strength of the biopotential signal acquired by the sensor.

14. The method of claim 12, wherein the method further comprises:
identifying, by the one or more processors, a number of sensors of the set of sensors that have no contact with the patient;
identifying, by the one or more processors, a number of sensors of the set of sensors that have loose contact with the patient; and
determining, by the one or more processors, whether a sum of the number of sensors that have no contact and the number of sensors that have loose contact is greater than a threshold number of sensors.

15. The method of claim 14, wherein based on determining that the number of sensors is greater than the threshold number of sensors, method further comprises:
causing, by the one or more processors, the second interactive page displayed by the user device to identify each sensor of the set of sensors that has no contact with the patient;
causing, by the one or more processors, the second interactive page displayed by the user device to identify each sensor of the set of sensors that has loose contact with the patient; and
causing, by the one or more processors, the second interactive page displayed by the user device to identify each sensor of the set of sensors that has good contact with the patient.

16. The method of claim 14, wherein based on determining that the number of sensors is not greater than the threshold number of sensors, the method further comprises:
causing, by the one or more processors, the user device to display a third interactive page corresponding to a performance of the physiological data collection procedure, wherein the third interactive page indicates an amount of time remaining in the physiological data collection procedure; and
controlling, by the one or more processors, the wearable device to perform the physiological data collection procedure by collecting one or more physiological signals via the set of sensors.

17. The method of claim 16, wherein when the physiological data collection procedure is complete, the method further comprises:
causing, by the one or more processors, the user device to display a fourth interactive page which indicates that results from the physiological data collection procedure is uploading for analysis; and
uploading, by the one or more processors, the results from the physiological data collection procedure to a fetal monitoring system for analysis.

18. The method of claim 11, further comprising:
receiving, by the one or more processors, a user input selecting the physiological data collection procedure from a set of physiological data collection procedures, wherein the set of physiological data collection procedures include a health check procedure and a nonstress test procedure; and
causing, by the one or more processors, the user device to display the first interactive page based on receiving the user input.

19. The method of claim 18, further comprising:
receiving, by the one or more processors, the user input selecting the health check procedure; and
controlling, by the one or more processors based on the level of contact between the patient and each sensor of the set of sensors, the wearable device to perform the health check procedure by collecting one or more physiological signals from the patient, wherein the one or more physiological signals indicate a maternal cardiac activity and a fetal cardiac activity.

20. A non-transitory computer-readable medium comprising instructions for causing one or more processors to:
receive, from a user device, a user input including a request to initiate a physiological data collection procedure;
cause the user device to display a first interactive page including one or more instructions for preparing for the physiological data collection procedure;
receive, from the user device, a user input including a request to start the physiological data collection procedure; and
cause the user device to display a second interactive page including a set of icons, wherein each icon of the set of icons corresponds to a sensor of a set of sensors on a wearable device of the patient, and wherein each icon of the set of icons indicates a level of contact between a patient and the respective sensor of the set of sensors.
